(19) United States
(12) Patent Application Publication
(10) Pub. No.: US 2026/0216517 A1
(43) Pub. Date: Jul. 30, 2026

(54) LOW DUTY-CYCLE CLOSED-LOOP CONTROL OF STIMULATION

(71) Applicant: Medtronic, Inc., Minneapolis, MN (US)

(72) Inventors: Julia P. Slopsema, Minneapolis, MN (US); Hank T. Bink, Golden Valley, MN (US); Lisa M. Jungbauer Nikolas, Lino Lakes, MN (US); Nicholas D. Buse, New Brighton, MN (US); Sarah J. Offutt, Golden Valley, MN (US); Simeng Zhang, Golden Valley, MN (US); Joshua O. Usoro, St. Paul, MN (US); Yao-Chuan Chang, New Brighton, MN (US)

(21) Appl. No.: 19/456,060

(22) Filed: Jan. 22, 2026

Related U.S. Application Data
(60) Provisional application No. 63/749,380, filed on Jan. 24, 2025.

(51) Int. Cl. A61N 1/36; A61N 1/372

(52) U.S. Cl. CPC A61N 1/36139; A61N 1/3615; A61N 1/36178; A61N 1/37241

(57) ABSTRACT

An example system includes processing circuitry configured to: control stimulation circuitry to deliver a therapy stimulation and a test stimulation via a plurality of electrodes, wherein the therapy stimulation is different than the test stimulation; control sensing circuitry to sense an evoked response elicited by the test stimulation during a sensing window following the test stimulation; and adjust, based on the evoked response, the therapy stimulation at a first cadence, wherein the first cadence is equal to or longer than once per hour.

This application claims the benefit of U.S. Provisional Patent Application No. 63/749,380, filed Jan. 24, 2025, the entire contents of which is incorporated herein by reference.

### TECHNICAL FIELD

This disclosure generally relates to electrical stimulation, and more specifically, to adjusting electrical stimulation over time.

### BACKGROUND

Medical devices may be external or implanted, and may sense electrical signals (e.g., neuromuscular signals from central and/or peripheral nerves/muscles) and/or deliver electrical stimulation therapy to various tissue sites of a patient to treat a variety of symptoms or conditions such as, for example, one or more of bladder dysfunction (e.g., retention, overactive bladder, urgency, urgency frequency, urinary incontinence, bladder incontinence, stress incontinence, nocturia, or any other dysfunction of the bladder), bowel dysfunction (e.g., fecal incontinence, intractable constipation, irritable bowel syndrome, inflammatory bowel disease, or any other dysfunction of the bowels), chronic pain, stroke, spinal cord injury, neuropathy, tremor, Parkinson's disease, multiple sclerosis, other movement disorders, epilepsy, sexual dysfunction, obesity, gastroparesis, pelvic pain, interstitial cystitis, sleep apnea, neural control of prosthetic devices, or stimulation to provide peripheral sensation. A medical device delivers electrical stimulation therapy via one or more leads that include electrodes located proximate to target locations associated with the brain, the spinal cord, pelvic nerves (e.g., sacral nerve stimulation, tibial nerve stimulation, saphenous nerve stimulation, pudendal nerve stimulation, dorsal genital nerve stimulation, inferior rectal nerve, perineal nerve, pudendal, dorsal genital, inferior rectal, or perineal nerve), peripheral nerves (e.g. tibial or saphenous nerve), or the gastrointestinal tract of a patient. For bipolar stimulation, the electrodes used for stimulation may be on one or more leads. For unipolar stimulation, the electrodes may include one or more leads and an electrode on a stimulator housing located remotely from the target site (e.g., near clavicle or near buttocks).

A clinician (or other healthcare provider) or patient may select values for a number of programmable parameters, via an external programmer, in order to define the electrical stimulation therapy to be delivered by the implantable stimulator to a patient. For example, the clinician may select one or more electrodes for delivery of the stimulation, a polarity of each selected electrode, a voltage or current pulse magnitude, a pulse width, and a pulse rate as stimulation parameters. A set of parameters, such as a set including electrode combination, electrode polarity, magnitude, pulse width, and pulse rate, may be referred to as a program in the sense that the parameters define the electrical stimulation therapy to be delivered to the patient.

### SUMMARY

This disclosure describes example techniques for processing circuitry configured to adjust one or more stimulation parameters automatically and/or control a user interface to notify a clinician or other user of a recommended change to one or more stimulation parameters. An implantable medical device (IMD) and/or an external programmer for the IMD may monitor one or more sensed electrical signals after implantation of the IMD. The sensed electrical signals may be evoked signals. The system may generate a metric from the sensed electrical signals and compare the metric to a threshold or threshold range. If the metric is outside of the range, the IMD may improve therapy by completing one or more actions including adjust one or more of the stimulation parameters and/or control a user interface to notify a clinician or other user of a recommended change to one or more stimulation parameters. The generation of the metric from the sensed electrical signals may occur at a relatively lower rate for closed-loop therapy, such as a frequency no greater than once per hour. Slower frequencies for sensing and/or adjustment may include frequencies such as once per day, once every five days, or other such frequencies.

In one example, this disclosure describes a system includes processing circuitry configured to control stimulation circuitry to deliver a therapy stimulation and a test stimulation via a plurality of electrodes, wherein the therapy stimulation is different than the test stimulation. The processing circuitry is further configured to control sensing circuitry to sense an evoked response elicited by the test stimulation during a sensing window following the test stimulation and adjust, based on the evoked response, the therapy stimulation at a first cadence, wherein the first cadence is equal to or longer than once per hour.

In another example, this disclosure describes a method includes controlling, by processing circuitry, stimulation circuitry to deliver a therapy stimulation and a test stimulation via a plurality of electrodes, wherein the therapy stimulation is different than the test stimulation. The method further includes controlling, by the processing circuitry, sensing circuitry to sense an evoked response elicited by the test stimulation during a sensing window following the test stimulation and adjusting, by the processing circuitry and based on the evoked response, the therapy stimulation at a first cadence, wherein the first cadence is equal to or longer than once per hour.

In another example, this disclosure describes a non-transitory computer-readable storage medium includes control stimulation circuitry to deliver a therapy stimulation and a test stimulation via a plurality of electrodes, wherein the therapy stimulation is different than the test stimulation. The non-transitory computer-readable storage medium further includes instructions to control sensing circuitry to sense an evoked response elicited by the test stimulation during a sensing window following the test stimulation and adjust, based on the evoked response, the therapy stimulation at a first cadence, wherein the first cadence is equal to or longer than once per hour.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver electrical stimulation to one or more pelvic nerves of a patient according to an example of the techniques of the disclosure.

FIG. 2 is a block diagram of the example IMD of FIG. 1 for delivering electrical stimulation according to an example of the techniques of the disclosure.

FIG. 3 is a block diagram of the external device of FIG. 1 for communicating with a medical device according to an example of the techniques of the disclosure.

FIG. 4 is a flowchart illustrating an example operation of a device configured to determine whether the processed signal is out of a range and adjusting stimulation in response to the processed signal being out of the range.

FIG. 5A is a flowchart illustrating an example operation of a device configured to process the sensed signal based on peak-to-peak averages between an evoked response and a baseline window.

FIG. 5B is graph of an example alternating polarity stimulation employing active recharge.

FIG. 5C is graph of an example alternating polarity stimulation employing passive recharge.

FIG. 6 is a flowchart illustrating an example operation of a device configured to sense one or more signals after exiting a standby mode and/or before entering the standby mode.

FIG. 7 is a flowchart illustrating an example operation of a device configured to adjust therapy at a frequency no greater than once an hour after comparing evoked responses to baseline responses.

FIG. 8 is a graph of example of sensed signals and adjusted stimulation magnitude over time.

### DETAILED DESCRIPTION

This disclosure describes example devices, systems, and techniques related to managing stimulation parameters delivered by a medical device. Currently, clinicians will set the stimulation parameters of the medical device and often only update the stimulation parameters at appointments. Typically, the appointments are three months after implantation, six months after implantation, and one year after implantation. After the one-year appointment, the clinician typically only updates the stimulation parameters annually (i.e., once a year). Patients may adjust a limited set of parameters at home, but often wait for appointments to have clinicians make the adjustments. However, the patient's therapy response may change (e.g., day to day, week to week, and month to month) and therefore stimulation parameters which provided suitable therapy for the patient, may be insufficient or uncomfortable between clinic visits. Therefore, a device, method, or system capable of measuring a physiological response of a patient and adjusting the stimulation parameters based on the response is desired to reduce the burden on clinicians and improve patient therapy.

A medical device may deliver a test-stimulation and sense a response evoked by the test-stimulation a set number of times per day. The system may detected an evoked response by detecting characteristics of the evoked response (e.g., peak detection above a threshold, peak detection relative to baseline noise (such as amplitude, standard deviation, etc.), or classification detection using techniques such as neural networks or decision trees) or comparing different portions of the evoked response (e.g., comparing an earlier window of the sensed signal that may be representative of a present evoked response to a later window of the sensed signal that may be representative of a baseline value). The medical device may compare one or more evoked responses to a threshold a set number of times per week. The medical device may adjust stimulation parameters based on the comparison a set number of times per week. Therefore, the medical device may measure the response of the patient and adjust the stimulation parameters based on the response such that the therapy delivered to the patient suitably adjusts a set number of times per week.

Advantages of sensing the evoked response the set number of times per day include reducing energy consumption compared to sensing the evoked response at a higher frequency. Reducing energy consumption in an energy constrained environment, such as an implantable medical device, is important to increase the longevity of the device and therefore reduces replacement (i.e., explanation and reimplantation) risks. Such advantages likewise apply to comparing the one or more evoked responses to the threshold and adjusting the stimulation parameters the set number of times per week. Specifically, reducing the number computations, i.e., comparisons and updates, that the medical device does decreases power consumption thereby increasing longevity. Furthermore, advantages of sensing the evoked response the set number of times per day may include reducing the frequency with which a patient could potentially feel the test stimulation. Advantages of adjusting the stimulation parameters the set number of times per week may further include reducing the number of patient visits to the clinician to adjust therapy, thereby reducing medical care costs for the patient and improving patient experience.

To measure the response of the patient to stimulation, the medical device may sense any signal of the patient via implanted electrodes. The medical device may be an implantable medical device configured to deliver electrical stimulation via the one or more implanted electrodes. The one or more implanted electrodes may be implanted proximate a sacral nerve of the patient. The sensed signal may be a physiological response to a test stimulation. The sensed signals may be one or more sensed signals with evoked via stimulation with active recharge. The sensed signal may be an Evoked Compound Action Potential (ECAP), an Electromyography (EMG) signal, any other signal evoked in response to the test stimulation, and/or any combination thereof. The medical device may measure the response of the patient to stimulation once an hour, once a day, or whenever the device exits a sleep mode. The medical device may compare the measured response to a threshold and adjust the parameters based on the response at the same or less frequently such as seven times a week (i.e., once a day), once every five days, or any other suitable frequency. The threshold may be a range and may be based on signal peak-peak magnitude of the evoked response (e.g. between 1 μV-10 mV). As a result of the evoked response being below the threshold range, stimulation may be increased and as a result of the evoked response being above the threshold range, stimulation may be decreased. The stimulation may be adjusted, i.e., increased or decreased, by 0.1 mA.

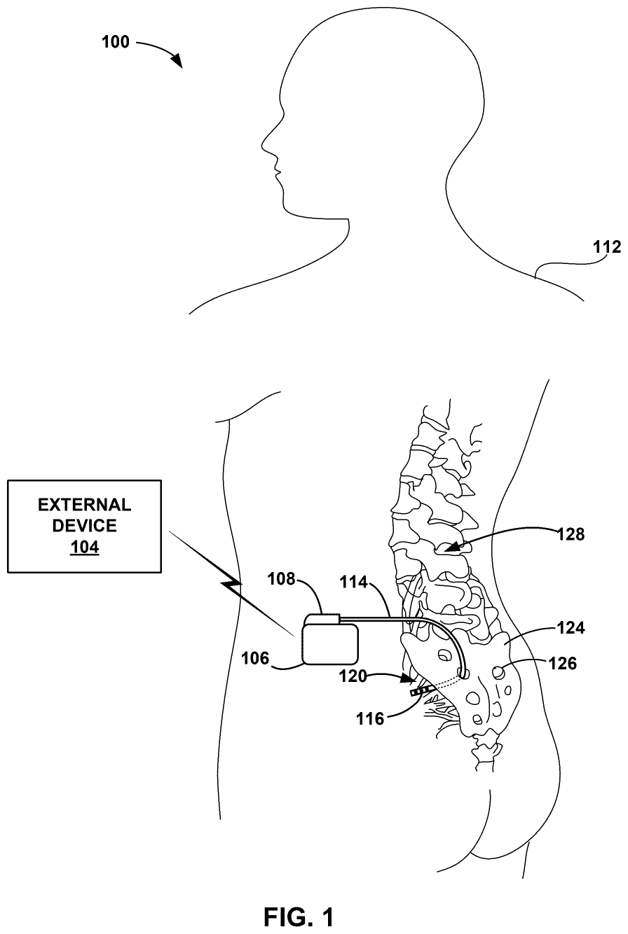

FIG. 1 is a conceptual diagram illustrating an example system 100 that includes implantable medical device (IMD) 106 which may deliver therapy to and/or sense physiological signals from target tissue. The target tissue may include or be near spinal cord 128 and/or pelvic nerves 120 (e.g., a sacral nerve or a pudendal nerve), dorsal genital nerve, perineal nerve, inferior rectal nerve, pudendal nerve, external anal sphincter muscle, coccygeus muscle, levator ani muscle group, bulbocavernosus and/or bulbospongiosus muscle, gluteal muscles, e.g., gluteus maximus, gluteus medius, and gluteus minimus, perineal muscles, ischiocavernosus muscles, puborectalis muscles, piriformis muscles, detrusor muscle, or any other muscles, or any other nervous or muscle tissue that may be stimulated or from which physiological signals may be sensed of patient 112 through lead 114 (coupled to IMD 106 via connector 108). Lead 114 may carry a plurality of electrodes 116 at the distal end of lead 114. IMD 106 may provide neurostimulation to treat symptoms of patient 112, such as fecal or urinary incontinence, pain,, erectile dysfunction, or other sexual dysfunction. IMD 106 may thus be configured to provide sacral nerve stimulation in one example.

Lead 114 may include one or more additional leads that may carry electrodes 118 (not shown in FIG. 1). Electrodes 118 may be substantially similar to electrodes 116. In some examples, some of electrodes 116, 118 are configured to sense signals and others of electrodes 116, 118 may be configured to deliver adaptive electrical stimulation to the target tissue. In other examples, all of electrodes 116, 118 are configured to both sense signals and deliver adaptive electrical stimulation to nerve 120. In some examples, unipolar stimulation is possible where one electrode is a housing or otherwise on the housing (i.e., a can) of IMD 106.

Although the examples described in this disclosure are generally applicable to a variety of medical devices including external devices and IMDs, application of such techniques to IMDs and, more particularly, implantable electrical stimulators (e.g., neurostimulators) are described for purposes of illustration. More particularly, the disclosure will refer to a sacral nerve stimulation (SNS) for purposes of illustration, but without limitation as to other types of medical devices or other therapeutic applications of stimulation. Specifically, although FIG. 1 is directed to sacral nerve stimulation (SNS), e.g., to treat pain, fecal or urinary incontinence, erectile dysfunction, or other sexual dysfunction, system 100 may be configured to treat any other condition that may benefit from electrical stimulation therapy. For example, system 100 may be configured to deliver SCS therapy, e.g., used to treat pain, and/or system 100 may be configured to deliver DBS therapy, e.g., used to treat obesity, gastroparesis, or psychiatric disorders (e.g., depression, mania, obsessive compulsive disorder, anxiety disorders, and the like). In this manner, system 100 may be configured to deliver one or more of sacral nerve stimulation (SNS), deep brain stimulation (DBS), spinal cord stimulation (SCS), tibial nerve stimulation (TNS), saphenous nerve stimulation, pelvic stimulation, pelvic floor stimulation, gastric stimulation, gastrointestinal stimulation, peripheral nerve field stimulation (PNFS), peripheral nerve field stimulation (PNFS), cortical stimulation (CS), or any other stimulation therapy capable of treating a condition of patient 112. Patient 112 ordinarily is a human patient. In some cases, however, therapy system 100 may be applied to other mammalian, non-mammalian, or non-human patients.

SNS, or other therapies such as DBS or TNS, may operate open loop or, alternatively, adaptive in the sense that IMD 106 may adjust, increase, or decrease the magnitude of one or more parameters of the SNS in response to changes in patient activity or movement, a severity of one or more symptoms of a disease of patient 112, a presence of one or more side effects due to the SNS, or one or more sensed signals of patient 112. For example, one example of system 100 is an SNS system with capabilities to both deliver stimulation and sense intrinsic neuronal signals. System 100 may provide for “closed-loop” therapy where IMD 106 may continuously monitor the state of certain biomarker signals and deliver stimulation according to pre-programmed routines based on the biomarker signals.

IMD 106 includes a therapy module (e.g., which may include processing circuitry, signal generation circuitry, or other electrical circuitry configured to perform the functions attributed to IMD 106) that includes a stimulation generator configured to generate and deliver electrical stimulation therapy to patient 112 via a subset of electrodes 116, 118 of lead 114. The subset of electrodes 116, 118 that are used to deliver electrical stimulation to patient 112, and, in some cases, the polarity of the subset of electrodes 116, 118 may be referred to as a stimulation electrode combination. The stimulation electrode combination may be selected for a particular patient 112 and target tissue site (e.g., selected based on the patient condition or selected based on the lead orientation with respect to nerve 120). The group of electrodes 116, 118 includes at least one electrode and may include a plurality of electrodes. In some examples, the plurality of electrodes 116, 118 have a complex electrode geometry such that two or more electrodes are located at different positions around the perimeter of the respective lead.

In some examples, sensed signals reflect changes in electrical current produced by the sum of electrical potential differences among nerves, such as nerve 120, in the region. Examples of neurological signals include, but are not limited to, bioelectric signals generated from local nerves and muscles sensed within one or more regions near nerve 120. In some examples, IMD 106 employs an electromyogram (EMG) to measure a muscle response or electrical activity in response to a nerve's stimulation of the muscle. In some examples, IMD 106 measures evoked compound action potentials (ECAPs) which may be a measure of the quantity and magnitude of one or more action potentials evoked by stimulation. An electroneurogram (ENG) may record the electrical activity of neurons of the central nervous system (brain and spinal cord) or the peripheral nervous system (nerves and ganglions). An EMG may involve placing electrodes in or proximate neural tissue to record the electrical signals generated by muscle tissue. In some examples, IMD 106 may sense evoked signals, such as electroneurograms (ENGs), evoked compound action potentials (ECAPs), evoked resonant neural activity (ERNA), electromyogram (EMG), etc., or any combination therefore. IMD may additionally sense baseline signals which are different than evoked signals. In some examples, IMD 106 may sense evoked signals in response to one or more stimulations provided by IMD 106. IMD 106 may provide stimulations which are bipolar or unipolar. IMD 106 may provide stimulations which include active recharge or passive recharge. IMD 106 may provide stimulations wherein the polarity alternates between two or more electrodes of electrodes 116, 118.

Electrical stimulation generated by IMD 106 may be configured to manage a variety of disorders and conditions. In some examples, the stimulation generator of IMD 106 is configured to generate and deliver electrical stimulation pulses to patient 112 via electrodes of a selected stimulation electrode combination. However, in other examples, the stimulation generator of IMD 106 may be configured to generate and deliver a continuous wave signal, e.g., a sine wave or triangle wave. In either case, a stimulation generator within IMD 106 may generate the electrical stimulation therapy for SNS according to a selected therapy program. In examples in which IMD 106 delivers electrical stimulation in the form of stimulation pulses, a therapy program may include a set of therapy parameter values (e.g., stimulation parameters), such as a stimulation electrode combination for delivering stimulation to patient 112, pulse frequency, pulse width, and a current or voltage magnitude of the pulses. As previously indicated, the electrode combination may indicate the specific electrodes 116, 118 that are selected to deliver stimulation signals to tissue of patient 112 and the respective polarities of the selected electrodes. The electrical stimulation generated by IMD 106 may generate, for example, burst pulses, interleaved pulses, or concurrent pulses.

In some examples, electrodes 116, 118 are radially-segmented electrodes. Radially-segmented electrodes refer to electrodes that are segmented radially along the lead. As one example, lead 114 may include a first set of electrodes 116, 118 arranged circumferentially around lead 114 that are all at the same height level on lead 114. Each of electrodes 116, 118 in the first set of electrodes 116, 118 is a separate segmented electrode and form a level of radially-segmented array of electrodes. Lead 114 may include a second set of electrodes arranged circumferentially around lead 114 that are all at the same height level on lead 114. Each of the electrodes 116, 118 in the first set of electrodes is a separate segmented electrode and form a level of radially-segmented array of electrodes. Segmented electrodes 116, 118 may be beneficial for directional stimulation and sensing.

IMD 106 may be implanted within a subcutaneous pocket, or at any other suitable site within patient 112. Generally, IMD 106 is constructed of a biocompatible material that resists corrosion and degradation from bodily fluids. IMD 106 may include a hermetic housing to substantially enclose components, such as a processor, therapy module, and memory.

System 100 may additionally include an implanted lead extension coupled to IMD 106 via connector 108 (also referred to as a connector block or a header of IMD 106). In the example shown in FIG. 1, lead 114 is implanted near a sacral region of the spinal cord of patient 112 in order to deliver electrical stimulation to one or more regions of nerve 120, which may be selected based on the patient condition or disorder controlled by therapy system 100. The specific target tissue site and the stimulation electrodes used to deliver stimulation to the target tissue site, however, may be selected, e.g., according to the identified patient responses (e.g., toe/bellow motor response) and/or other sensed patient parameters (EMG/ECAP). For example, the target tissue site may be the location of bioelectric signals includes a signal component of interest. One or more lead 114 and/or IMD 106 implant sites are suitable depending on clinical application or target tissue/nerve.

Existing lead sets include axial leads carrying ring electrodes disposed at different axial positions and so-called “paddle” leads carrying planar arrays of electrodes. In some examples, lead 114 includes electrode combinations within an axial lead, a paddle lead, or among two or more different leads. Alternatively, more complex lead array geometries may be used. Electrodes 116, 118 may additionally or alternatively be cuff electrodes wrapped around the sacral (or other pelvic) nerve.

Lead 114 may be coupled to a common lead extension (not shown) wherein the common lead extension extends from connector 108 to any point closer to the implant site or region of interest, wherein one or more leads 114 may be connected to the common lead extension. The common lead extension enables IMD 106 to be implanted at a site distal to the region of interest while enabling the one or more leads 114 to extend a shorter distance than they would otherwise extend. In other examples, one or more leads 114 may be coupled to IMD 106 via separate lead extensions or directly to connector 108. Lead 114 may be positioned to deliver electrical stimulation to one or more target tissue sites within nerve 120 to manage patient symptoms associated with a movement disorder of patient 112. Lead 114 may be implanted to position electrodes 116, 118 at suitable locations of nerve 120 through respective holes in sacrum 124. Leads 114 may be placed at any location along nerve 120 such that electrodes 116, 118 are capable of providing electrical stimulation to target tissue sites along nerve 120 during treatment. For example, electrodes 116, 118 may be surgically implanted by inserting lead 114 through sacral foramina 126 of sacrum 124 of patient 112. Specifically, lead 114 may be inserted from a dorsal side of sacrum 124 through a sacral foramen to a ventral side of sacrum 124. Lead 114 may be inserted into the S3 sacral foramen such that electrodes extend along nerve 120 without manipulation of lead 114 on a ventral side of sacrum 124. Electrodes 116, 118 may be electrically coupled to IMD 106 via one or more leads 114. In some examples, lead 114 may be inserted into other foramen, such as S1, S2, S4 or S5. Lead 114 may be implanted to position electrodes 116, 118 at suitable locations of a pudendal nerve or a tibial nerve of patient 112. Leads 114 may be placed at any location along the pudendal nerve or the tibial nerve such that electrodes 116, 118 are capable of providing electrical stimulation to target tissue sites along the pudendal nerve or the tibial nerve during treatment. For example, electrodes 116, 118 may be surgically implanted by inserting lead 114 proximate to the pudendal nerve or the tibial nerve. Specifically, lead 114 may be inserted such that electrodes extend along the pudendal nerve or the tibial nerve to deliver the therapy stimulation. Electrodes 116, 118 may be electrically coupled to IMD 106 via one or more leads 114. The target location may comprise at least one of the pudendal nerve or the tibial nerve of the patient.

In the example shown in FIG. 1, electrodes 116, 118 of leads 114 are shown as ring electrodes. Ring electrodes may be used in SNS applications because ring electrodes are relatively simple to program and are capable of delivering an electrical field to any tissue adjacent to electrodes 116, 118. In other examples, electrodes 116, 118 may have different configurations. For example, at least some of electrodes 116, 118 of leads 114 may have a complex electrode array geometry that is capable of producing shaped electrical fields. The complex electrode array geometry may include multiple electrodes (e.g., partial ring or segmented electrodes) around the outer perimeter of each lead 114, rather than one ring electrode. In this manner, electrical stimulation may be directed in a specific direction from leads 114 to enhance therapy efficacy and reduce possible adverse side effects from stimulating a large volume of tissue.

In some examples, a housing of IMD 106 includes one or more stimulation and/or sensing electrodes. The housing of IMD 106 may additionally or alternatively be referred to as a can. The can may operate as an electrode, by providing stimulation and/or sensing through the surface of the device itself. In some examples, leads 114 have shapes other than elongated cylinders as shown in FIG. 1. For example, lead 114 may be paddle leads, spherical leads, bendable leads, or any other type of shape effective in treating patient 112 and/or minimizing invasiveness of lead 114.

IMD 106 includes a memory to store a plurality of therapy programs that each define a set of therapy parameter values. In some examples, IMD 106 selects a therapy program from the memory based on various parameters, such as sensed patient parameters and the identified patient behaviors. IMD 106 may generate electrical stimulation based on the parameters of the selected therapy program to manage the patient symptoms associated with a pelvic health disorder.

External device 104 wirelessly communicates with IMD 106 as suitable to provide or retrieve therapy information. External device 104 is an external computing device that the user, e.g., a clinician and/or patient 112, may use to communicate with IMD 106. For example, external device 104 may be a clinician programmer that the clinician (e.g., doctor, physician, nurse) uses to communicate with IMD 106 and program one or more therapy programs for IMD 106. Alternatively, external device 104 may be a patient programmer that allows patient 112 to select programs and/or view and modify therapy parameters. The clinician programmer may include more programming features than the patient programmer. In other words, more complex or sensitive tasks may only be allowed by the clinician programmer to reduce or limit an untrained patient from making changes to IMD 106. External device 104 may be any type of computing device, such as a proprietary device, a cellular phone, a smartphone, a tablet computing device, a laptop, or any other type of computing device. Generally, external device 104 includes a user interface that may provide and/or receive information from a user. In some examples, external device 104 is configured to pass information between IMD 106 and a different external device, but external device 104 may or may not have a user interface for programming. Instead, external device 104 may receive programming commands from the different external device (e.g., a server or other computing device that includes a user interface) and transmit those programming commands to IMD 106 and/or receive information from IMD 106 and send that information to the different external device.

When external device 104 is configured for use by the clinician, external device 104 is configured to transmit initial programming information to IMD 106. This initial information may include hardware information, such as the type of leads 114 and the electrode arrangement, the position of leads 114 adjacent to nerve 120, the configuration of electrode array 116, 118, initial programs defining therapy parameter values, and any other information the clinician suitably programs into IMD 106. External device 104 may also be capable of completing functional tests (e.g., measuring the impedance of electrodes 116, 118 of lead 114).

The clinician may also store therapy programs within IMD 106 with the aid of external device 104. During a programming session, the clinician may determine one or more therapy programs that may provide efficacious therapy to patient 112 to address symptoms associated with the patient condition, and, in some cases, specific to one or more different patient states, such as a sleep state, movement state or rest state. For example, the clinician may select one or more stimulation electrode combinations with which stimulation is delivered to nerve 120 and such stimulation electrode combinations may change based on the one or more different patient states and additionally may be continuously or intermittently updated by IMD 106. During the programming session, the clinician may evaluate the efficacy of the specific program being evaluated based on feedback provided by patient 112 or based on one or more sensed physiological parameters of patient 112. External device 104 may assist the clinician in the creation/identification of therapy programs by providing a methodical system for identifying potentially beneficial therapy parameter values and/or identifying which electrodes 116, 118 to deliver stimulation from and/or which electrodes 116, 118 to sense signals.

However, in some examples, IMD 106 or external device 104 (e.g., a clinician programmer, a patient programmer, a recharger, a programmer fob, any electronic device suitable to install a therapy application, a smartphone, personal computing device, etc.), alone or in combination, may automatically determine electrode configuration and therapy parameters. In some examples, external device 104 outputs information indicating the selected electrode configuration for stimulation and the determined stimulation magnitude or other therapy parameter for the clinician (e.g., a clinician or a physician) to review and confirm before IMD 106 delivers therapy via the selected electrode configuration with the determined stimulation magnitude. In some examples, external device 104 outputs information indicating the selected electrode configuration for sensing. External device 104 or IMD 106 may additionally or alternatively automatically adjust the stimulation parameters, the stimulation electrodes, and/or the sensing electrodes based on one or more criteria. The one or more criteria may be preset by a clinician.

External device 104 may also be configured for use by patient 112. When configured as a patient programmer, external device 104 may have limited functionality (compared to a clinician programmer) in order to reduce or limit patient 112 from altering critical functions of IMD 106 or applications that may be detrimental to patient 112. In this manner, external device 104 may only allow patient 112 to adjust values for certain therapy parameters or set an available range of values for a particular therapy parameter. For example, external device 104 may only allow patient 112 to adjust a magnitude or an intensity (by combination or magnitude, pulse width and/or pulse rate).

External device 104 may also provide an indication to patient 112 when therapy is being delivered, when patient input has triggered a change in therapy or when the power source within external device 104 or IMD 106 needs to be replaced or recharged. For example, external device 104 may include an alert LED and/or a touchscreen, may flash a message to patient 112 via a programmer display, generate an audible sound or somatosensory cue to confirm patient input was received, e.g., to indicate a patient state or to manually modify a therapy parameter.

Therapy system 100 may be implemented to provide chronic stimulation therapy to patient 112 over the course of several months or years. However, system 100 may also be employed on a trial basis to evaluate therapy before committing to full implantation. If implemented temporarily, some elements of system 100 may not be implanted within patient 112. For example, patient 112 may be fitted with an external medical device, such as a trial stimulator, rather than IMD 106. The external medical device may be coupled to percutaneous leads or to implanted leads via a percutaneous extension. If the trial stimulator indicates SNS system 100 provides effective treatment to patient 112, the clinician may implant a chronic stimulator within patient 112 for relatively long-term treatment.

In some examples, IMD 106 is configured to provide electrical stimulation for treatment of a patient condition supplemental to medication provided to patient 112. Although some examples are described with the use of IMD 106 that provides stimulation, the techniques are not limited and the techniques may apply to examples where no stimulation is provided.

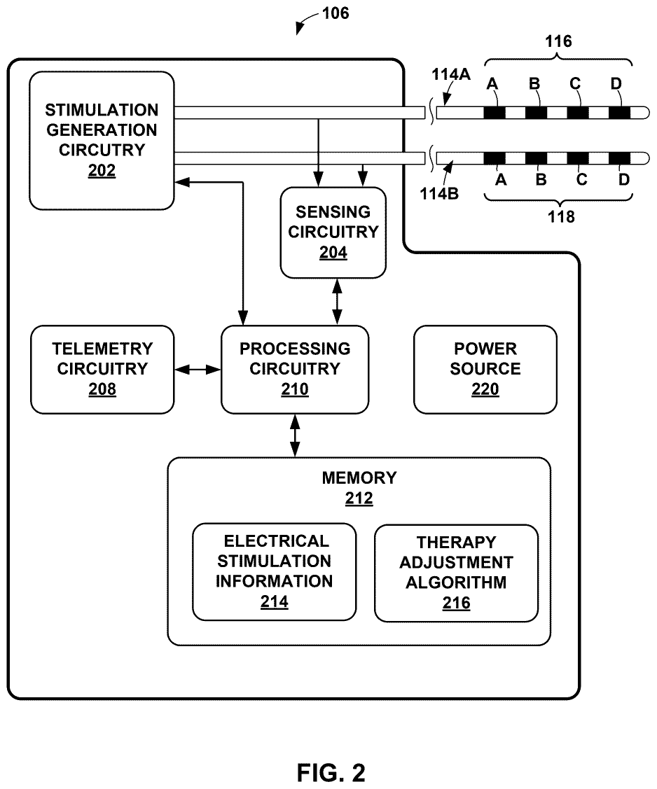

FIG. 2 is a block diagram of the example IMD 106 of FIG. 1 for delivering electrical stimulation according to an example of the techniques of the disclosure. In the example shown in FIG. 2, IMD 106 includes stimulation generation circuitry 202, sensing circuitry 204, telemetry circuitry 208, processing circuitry 210, memory 212, and power source 220. Each of these circuits may be or otherwise include electrical circuitry configured to perform the functions attributed to each respective circuit. Memory 212 may include any volatile or non-volatile media, such as a random-access memory (RAM), read only memory (ROM), non-volatile RAM (NVRAM), ferroelectric RAM (FRAM), electrically erasable programmable ROM (EEPROM), flash memory, and the like. Memory 212 may store computer-readable instructions (e.g., electrical stimulation information 214 and electrode selection program 216) that when executed by processing circuitry 210, cause IMD 106 to perform various functions. Memory 212 may be a storage device or other non-transitory medium.

Stimulation generation circuitry 202 may be a single channel or multi-channel stimulation generator. In particular, stimulation generation circuitry 202 may be capable of delivering a single stimulation pulse, multiple stimulation pulses, or a continuous signal at a given time via a single electrode combination or multiple stimulation pulses at a given time via multiple electrode combinations. Stimulation generation circuitry 202 may include multiple voltage or current sources and sinks that are coupled to respective electrodes to drive the electrodes as cathodes or anodes simultaneously or at different times. IMD 106 may or may not utilize switch circuitry for time-interleaved multiplexing of stimulation via different electrodes. Switch circuity may enable stimulation generation circuitry 202 to be configured to deliver multiple channels on a time-interleaved basis. Switch circuitry may serve to time divide the output of stimulation generation circuitry 202 across different electrode combinations at different times to deliver multiple programs or channels of stimulation energy to patient 112.

Processing circuitry 210 of IMD 106 may control sensing circuitry 204 to sense, via electrodes 116, 118 interposed along leads 114, one or more bioelectric signals of nerve 120 of patient 112. Processing circuitry 210 of IMD 106 may deliver, via electrodes 116, 118 (and stimulation generation circuitry 202), electrical stimulation therapy to patient 112 based on the sensed one or more bioelectric signals of nerve 120. Processing circuitry 210 of IMD 106 may select which electrodes of electrodes 116, 118 to connect to stimulation circuitry 202 and which electrodes of electrodes 116, 118 to connect to sensing circuitry 204 based on the sensed one or more bioelectric signals of nerve 120.

Telemetry circuitry 208 supports wireless communication using one or more communication protocols (e.g., using Bluetooth™, Wi-Fi™, Near-Field Communication (NFC), Near Field Magnetic Induction (NFMI), Long Term Evolution, 5th generation (LTE/5G), or MedRadio (MICS: Medical Implant Communication Service, MEDS: Medical External Device Service, MBAD: Medical Body Area Network)) between IMD 106 and an external device 104 or another computing device under the control of processing circuitry 210. In some examples, telemetry circuitry 208 supports a telemetry frequency that corresponds to a high frequency or radio frequency, which may be a radio frequency established via Bluetooth, Wi-Fi, Near-Field Communication (NFC), 175 KHz inductive telemetry, or MICS, for example. Telemetry circuitry 208 may be configured to receive an inductive sting. Processing circuitry 210 of IMD 106 may receive, as updates to programs (e.g., at least one program parameter), values for various stimulation parameters such as magnitude and electrode combination, from external device 104 via telemetry circuitry 208. The updates to the therapy programs may be stored within therapy programs 214 portion of memory 212. Telemetry circuitry 208 in IMD 106, as well as telemetry modules in other devices and systems described herein, such as external device 104, may accomplish communication by radiofrequency (RF) communication techniques (e.g., Bluetooth, Wi-Fi, Near-Field Communication (NFC), or MICS). In addition, telemetry circuitry 208 may communicate with external medical device external device 104 via proximal inductive interaction of IMD 106 with external device 104. Accordingly, telemetry circuitry 208 may send information to external device 104 on a continuous basis, at periodic intervals, or upon request from IMD 106 or external device 104.

Telemetry circuitry 208 may periodically output an advertisement packet for a connection at an advertising interval. The advertisement packet may include information on how to connect with the advertising device, such as, for example, one or more of: (1) media access control (MAC) addresses for the medical device and external device; (2) a real time-point in time for the transfer to start; (3) an indication of a starting frequency; (4) an indication of a hop set; (5) a connection interval; or (6) a connection latency. Telemetry circuitry 208 may be configured to output an advertisement packet, such as, an advertisement for a wireless communication session or advertisement compliant with another protocol.

Processing circuitry 210 may include fixed function processing circuitry and/or programmable processing circuitry, and may include, for example, any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 210 herein may be embodied as firmware, hardware, software or any combination thereof. Processing circuitry 210 may control stimulation generation circuitry 202 according to therapy programs 214 stored in memory 212 to apply particular stimulation parameter values specified by one or more of programs, such as voltage magnitude or current magnitude, pulse width, and/or pulse rate.

In the example shown in FIG. 2, the set of electrodes 116, 118 includes electrodes 116A, 116B, 116C, 116D, 118A, 118B, 118C, and 118D. Processing circuitry 210 may control individual voltage or current sources and sinks coupled to respective electrodes 116, 118, functioning as cathodes or anodes, to deliver stimulation signals to tissue of a patient, such as patient 112. In other examples, processing circuitry may control switch circuitry to apply the stimulation signals generated by stimulation generation circuitry 202 to selected combinations of electrodes 116, 118. In some examples, there may be eight electrodes in electrodes 116, 118. In other examples, there may be six, 10, 12, 16, 20, 24, or any other number, even or odd, of electrodes suitable for delivering stimulation to one or more locations of patient 112.

Power source 220 delivers operating power to various elements of IMD 106. Power source 220 may include a small rechargeable or non-rechargeable battery and a power generation circuit to produce the operating power. Recharging may be accomplished through proximal inductive interaction between an external charger and an inductive charging coil within IMD 106. In some examples, power consumption is small enough to allow IMD 106 to utilize patient motion and implement a kinetic energy-scavenging device to trickle charge a rechargeable battery. In other examples, traditional batteries may be used for a limited period of time.

In some examples, processing circuitry 210 continuously measures the one or more bioelectric signals in real time. In other examples, processing circuitry 210 may periodically sample the one or more bioelectric signals according to a predetermined frequency, external triggering event, or after a predetermined amount of time. In some examples, the predetermined amount of time may be once an hour, once every four hours, once every 24 hours, once a week, monthly, or any suitable duration between testing to sense changes of tissue proximate electrodes 116, 118. In some examples, processing circuitry 210 periodically samples the signal at a frequency of approximately 2-35 Kilohertz (kHz). In some examples, the sampling frequency may be 21.875 kHz, 25 kHz, 30 kHz, 20 kHz, or any other frequency suitable to record the response of tissue proximate electrodes 116, 118.

In the example shown in FIG. 2, memory 212 stores electrical stimulation information 214. Electrical stimulation information 214 may include program parameters (e.g., a therapy parameter set), such as a stimulation electrode combination, electrode polarity, current or voltage magnitude, pulse width, and pulse rate. In some examples, individual therapy programs are stored as a therapy group, which defines a set of therapy programs with which stimulation may be generated during a therapy session in which stimulation therapy is delivered. The stimulation signals defined by the therapy programs of the therapy group may be delivered together on an overlapping or non-overlapping (e.g., time-interleaved) basis.

Accordingly, in some examples, stimulation generation circuitry 202 generates electrical stimulation signals in accordance with the electrode stimulation information 214 stored in memory 212. Electrode stimulation information 214 may include the electrical stimulation parameters (e.g., program parameters) noted above. Other ranges of therapy parameter values may also be useful and may depend on the target stimulation site within patient 112. While stimulation pulses are described, stimulation signals may be of any form, such as continuous-time signals (e.g., sine waves) or the like. Stimulation generation circuitry 202 is an example of therapy circuitry configured to deliver a therapy from IMD 106. Other types of therapies may additionally, or alternatively, be delivered using therapy circuitry and IMD 106.

The adaptive therapy is defined by electrical stimulation information 214. For example, electrical stimulation information 214 may include a current magnitude (for a current-controlled system), a voltage magnitude (for a voltage-controlled system), a pulse rate or frequency, a pulse width, a number of pulses per cycle, an electrode selection, a program selection, or a schedule of therapy delivery (e.g. 30 min ON every 24 hours). In some examples, the electrode selection may modify which electrodes of electrodes 116, 118 are being used for stimulation and which electrodes of electrodes 116, 118 are being used for sensing. In examples where the electrical stimulation is delivered according to a “burst” of pulses, or a series of electrical pulses defined by an “on-time” and an “off-time,” the one or more parameters may define one or more of a number of pulses per burst, an on-time, and an off-time. Processing circuitry 210, via electrodes 116, 118, delivers to patient 112 adaptive SNS and may adjust one or more parameters defining the electrical stimulation based on corresponding parameters of the sensed one or more bioelectric signals of nerve 120.

In accordance with the techniques of the disclosure, processing circuitry 210 of IMD 106 may implement therapy adjustment algorithm 216 stored on memory 212 to adjust one or more electrical stimulation parameters stored in electrical stimulation information 214. Techniques described herein may including processing circuitry 210 of IMD 106 implementing therapy adjustment algorithm 216 to select one or more electrodes, deliver a test stimulus, sense an evoked response, and sense a baseline response and compare the evoked response and the baseline response. Techniques described herein may include processing circuitry 210 of IMD 106 implementing therapy adjustment algorithm 216 to adjust one or more parameters of electrical stimulation information 214 based on the comparison of the baseline response and the evoked response and/or control a user interface to notify a clinician, or any other user, that one or more parameters of electrical stimulation information 214 is recommended to be adjusted. Such automatic adjustments may increase the speed with which devices are adjusted to their patients and may decrease the time clinicians spend adjusting these parameters. When such adjustments occur less than once per hour, power savings are significant when compared with frequent adjustments.

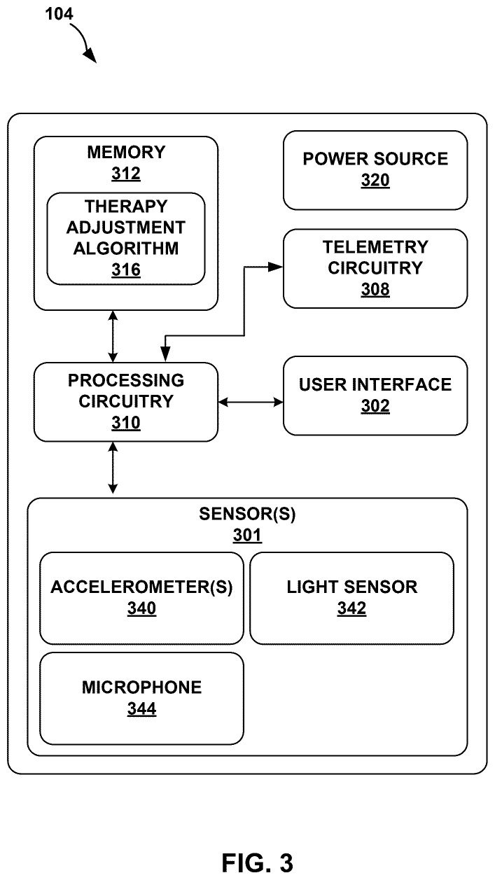

FIG. 3 is a block diagram of the external device 104 of FIG. 1. Although external device 104 may generally be described as a hand-held device, external device 104 may be a larger portable device or a stationary device. In addition, in other examples, external device 104 may be included as part of an external charging device or include the functionality of an external charging device. As illustrated in FIG. 3, external device 104 may include processing circuitry 310, memory 312, user interface 302, telemetry circuitry 308, and power source 320. Memory 312 may store instructions that, when executed by processing circuitry 310, cause processing circuitry 310 and external device 104 to provide the functionality ascribed to external device 104 throughout this disclosure. Each of these components, or modules, may include electrical circuitry that is configured to perform some or all of the functionality described herein. For example, processing circuitry 310 may include processing circuitry configured to perform the processes discussed with respect to processing circuitry.

In general, external device 104 includes any suitable arrangement of hardware, alone or in combination with software and/or firmware, to perform the techniques attributed to external device 104, and processing circuitry 310, user interface 302, and telemetry circuitry 208 of IMD 106. In various examples, external device 104 may include one or more processors, which may include fixed function processing circuitry and/or programmable processing circuitry, as formed by, for example, one or more microprocessors, DSPs, ASICs, FPGAs, or any other equivalent integrated or discrete logic circuitry, as well as any combinations of such components. External device 104 also, in various examples, may include a memory 312, such as RAM, ROM, PROM, EPROM, EEPROM, flash memory, a hard disk, a CD-ROM, comprising executable instructions for causing the one or more processors to perform the actions attributed to them. Moreover, although processing circuitry 310 and telemetry circuitry 308 are described as separate modules, in some examples, processing circuitry 310 and telemetry circuitry 308 are functionally integrated with one another. In some examples, processing circuitry 310 and telemetry circuitry 308 correspond to individual hardware units, such as ASICs, DSPs, FPGAs, or other hardware units. While telemetry circuitry 308 is described as being arranged within external device 104, in some examples, aspects of telemetry circuitry 308 (e.g., configuring a medical device to advertise at an advertising interval or initiating a communication session) may be performed by telemetry circuitry 308 external to external device 104 (e.g., in an intermediate device).

Memory 312 (e.g., a storage device) may store instructions that, when executed by processing circuitry 310, cause processing circuitry 310 and external device 104 to provide the functionality ascribed to external device 104 throughout this disclosure. For example, memory 312 may include instructions that cause processing circuitry 310 to obtain a parameter set from memory or receive a user input and send a corresponding command to IMD 106, or instructions for any other functionality. In addition, memory 312 may include a plurality of programs, where each program includes a parameter set that defines stimulation therapy. Memory 312 may store therapy adjustment algorithm 316 that, when executed by processing circuitry 310, cause processing circuitry 310 and external device 104 to perform one or more of the analyses, adjustments, changes, notifications, or other processes as described with regard to the below figures.

User interface 302 may include a button or keypad, lights, a speaker for voice commands, a display, such as a liquid crystal (LCD), light-emitting diode (LED), or organic light-emitting diode (OLED). In some examples the display is a touch screen. User interface 302 may be configured to display any information related to the delivery of stimulation therapy, identified patient behaviors, sensed patient parameter values, patient behavior criteria, or any other such information. User interface 302 may also receive user input. The input may be, for example, in the form of pressing a button on a keypad or selecting an icon from a touch screen. In some examples, the touch screen may be a touch screen of a user's own device (e.g., a smartphone, tablet, computer, etc.) with a therapy application.

One or more sensors 301 may include one or more accelerometers 340, a light sensor 342, and a microphone 344. For example, one or more accelerometers 340 may be configured to determine information indicating a movement of external device 104. Information may include one or more of an acceleration in an x-direction, acceleration in a y-direction, or an acceleration in a z-direction. The x-direction may be perpendicular to both the y-direction and the z-direction. External device 104 may additionally, or alternatively, include a gyroscope that may detect the movement and/or generate motion information. Light sensor 342 may be configured to determine light information (e.g., an ambient light level of an environment detected by light sensor 342). Microphone 344 may be configured to determine sound information (e.g., an ambient sound level of an environment detected by microphone 344). For example, microphone 344 may detect speech (e.g., from patient 112 or a caretaker of patient 112) and/or may detect bathroom usage.

Telemetry circuitry 308 may support wireless communication between IMD 106 and external device 104 under the control of processing circuitry 310. Telemetry circuitry 308 may also be configured to communicate with another computing device via wireless communication techniques, or direct communication through a wired connection. In some examples, telemetry circuitry 308 provides wireless communication via an RF or proximal inductive medium. In some examples, telemetry circuitry 308 includes an antenna (e.g., an internal or external antenna).

Examples of local wireless communication techniques that may be employed to facilitate communication between external device 104 and IMD 106 include RF communication according to the 802.11 or Bluetooth specification sets (e.g., Classic Bluetooth, Bluetooth high speed and Bluetooth Low Energy (BLE) protocols) or other standard or proprietary telemetry protocols. In this manner, other external devices may be capable of communicating with external device 104 without needing to establish a secure wireless connection. While examples described herein may refer to connections as using the Bluetooth protocol for establishing a connection and using advertisements compliant with the Bluetooth protocol other known and future protocols may be used. For example, techniques described herein for establishing a connection between IMD 106 and external device 104 may be compliant with any RF communication protocol and/or may use any telemetry frequency.

Telemetry circuitry 308 may receive the advertisement packet from IMD 106, for example, and connect with another device (e.g., IMD 106) using the received advertisement packet. The advertisement packet may include information on how to connect with the advertising device, such as, for example, one or more of: (1) media access control (MAC) addresses for the medical device and external device; (2) a real time-point in time for the transfer to start; (3) an indication of a starting frequency; (4) an indication of a hop set; (5) a connection interval; or (6) a connection latency.

In some examples, processing circuitry 310 defines the parameters of electrical stimulation therapy, stored in memory 312, for delivering adaptive SNS to patient 112. In one example, processing circuitry 310 of external device 104, via telemetry circuitry 308, issues commands to IMD 106 causing IMD 106 to deliver electrical stimulation therapy via electrodes 116, 118 via leads 114.

In accordance with the techniques of the disclosure, processing circuitry 210 of external device 104 may implement therapy adjustment algorithm 316 stored on memory 312 to adjust one or more electrical stimulation parameters. Techniques described herein may include processing circuitry 310 of external device 104 implementing therapy adjustment algorithm 316 to control IMD 106 to select one or more electrodes, deliver a test stimulus, sense an evoked response, and sense a baseline response and compare the evoked response and the baseline response. Techniques described herein may include processing circuitry 310 of external device 104 implementing therapy adjustment algorithm 316 to control IMD 106 to adjust one or more parameters of electrical stimulation information 214 based on the comparison of the baseline response and the evoked response and/or control user interface 302 to notify a clinician, or any other user, that one or more parameters of electrical stimulation information 214 is recommended to be adjusted. Such automatic adjustments may increase the speed with which devices are adjusted to their patients and may decrease the time clinicians spend adjusting these parameters. When such adjustments occur less than once per hour, power savings are significant when compared with frequent adjustments without decreasing the effectiveness of stimulation received by patient 112.

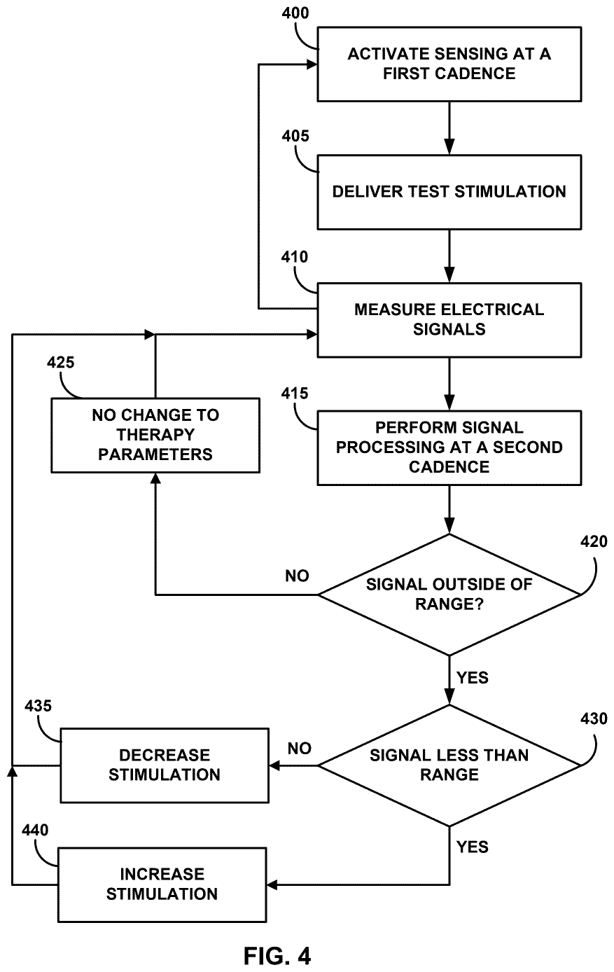

FIG. 4 is a flowchart illustrating an example operation of a device configured to determine whether the processed signal is out of a range and adjusting stimulation in response to the processed signal being out of the range. The example process of FIG. 4 will be described with respect to processing circuitry 210 of IMD 106, but other processing circuitry, such as processing circuitry 310 of external device 104 may additionally, or alternatively, perform at least some of the elements of the process.

The operation includes activating sensing at a first cadence (400), delivering test stimulation (405), measuring electrical signals (410), performing signal processing at a second cadence (415), determining whether the signals from the signal processing are outside of a range (420), and determining whether the signal is less than a range (430). If the signals are not outside the range, there is no change to therapy parameters (425), if the signals are outside the range and not less than the range (i.e., greater than the range) then decrease a stimulation intensity (435) and if the signals are outside the range and less than the range (i.e., less than the range) then increase a stimulation intensity (440).

A clinician may activate the implantable medical device prior to the operation of the device being configured to determine whether the processed signal is out of a range. The medical device may be activated with pre-set values provided by the manufacturer wherein the physician accepts the pre-set values wholesale or adjusts one or more of the parameters. The pre-set values may include, for example, one or more parameters of the stimulation such as one or more electrodes for delivery of the stimulation, a polarity of each selected electrode, a voltage or current pulse magnitude, a pulse width, a pulse rate as stimulation parameters, electrode combination, electrode polarity, magnitude, pulse width, and pulse rate. The pre-set values may include, for example, closed-loop therapy limits, i.e., the range of values for steps 420 and 430, as well as the magnitude by which to adjust one or more of the stimulation parameters in steps 435 and 440. The medical device may additionally or alternatively be activated with one or more values as set by the clinician. The medical device may have been activated at any prior time and may be reactivated at any later time.

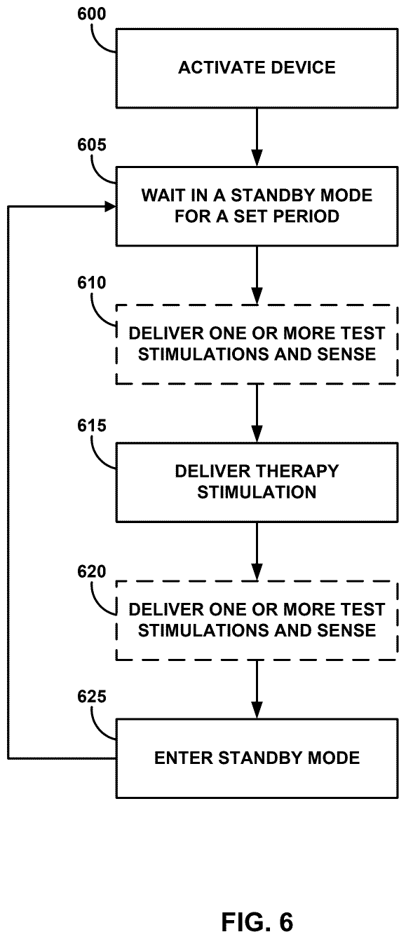

At a first cadence, processing circuitry 210 of IMD 106 may activate sensing circuitry 204 to sense signals from electrodes 116, 118 at a first cadence (400). Processing circuitry 210 may control sensing circuitry 204 to activate sensing upon exiting a sleep mode, as seen in FIG. 6. Processing circuitry 210 may control sensing circuitry 204 to activate sensing prior to entering a sleep mode, as additionally seen in FIG. 6. The electrical signals measured at the first cadence may be measured, or sensed, within a sensing window that occurs at the first cadence. The sensing window may include the test stimulation (e.g., one or more pulses) configured to elicit the signals that are measured. Specifically, processing circuitry 210 of IMD 106 may control stimulation generation circuitry 202 to deliver a stimulation signal via a plurality of electrodes 116, 118 to a target tissue (405). Processing circuitry 210 may control stimulation generation circuitry 202 to deliver a first pulse of a plurality of pulses at a selected magnitude or with one or more selected parameters. The one or more selected parameters may be any parameters as described throughout this disclosure.

Thereafter processing circuitry 210 of IMD 106 may control sensing circuitry 204 to measure electrical signals from a plurality of electrodes 116, 118. As such, processing circuitry 210 of IMD 106 may measure electrical signals (410). Processing circuitry 210 may then control sensing circuitry 204 to sense the stimulation-evoked signals in response to the first pulse of the plurality of pulses at the selected magnitude. Processing circuitry 210 may repeat steps 400-410 by controlling stimulation generation circuitry 202 and sensing circuitry 204 to repeat the process of activating sensing and delivering a pulse from the plurality of pulses at the selected magnitude until the first cadence has passed whereafter processing circuitry 210 proceeds to step 415. Processing circuitry 210 may repeat steps 400-410 by controlling stimulation generation circuitry 202 and sensing circuitry 204 to repeat the process of activating sensing and delivering a pulse from the plurality of pulses at the selected magnitude until sensing circuitry 204 has recorded a response from each stimulation pulse of the plurality of pulses. Processing circuitry 210 may additionally or alternatively repeat steps 400-410 by controlling stimulation generation circuitry 202 and sensing circuitry 204 to repeat the process of activating sensing and delivering a pulse from the plurality of pulses at subsequently increasing magnitudes, i.e., a ramp of magnitudes, until sensing circuitry 204 has recorded a suitable response. Stimulation generation circuitry 202 delivering a pulse prior to sensing generates a response for sensing circuitry 204 to record.

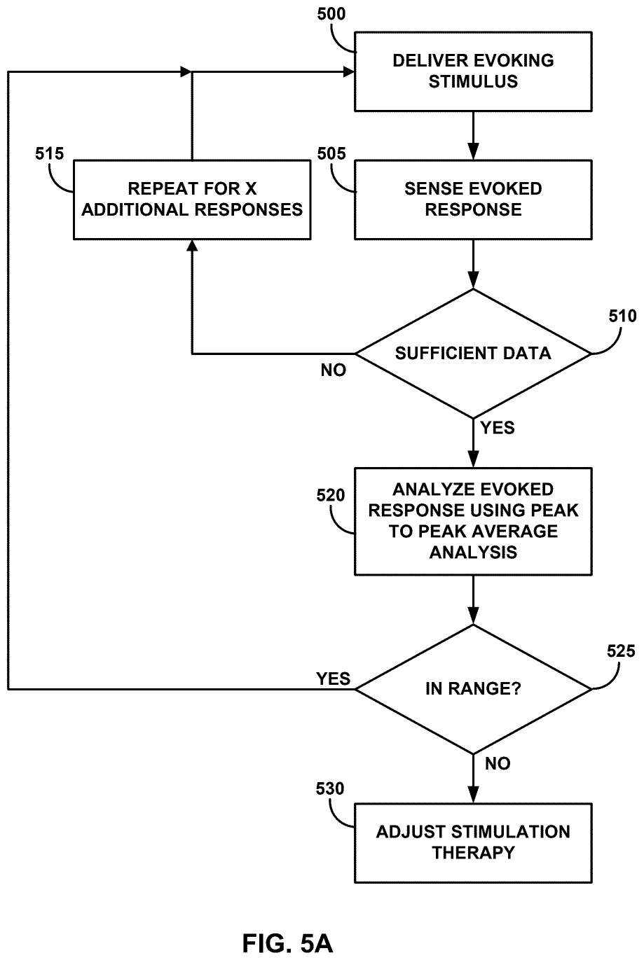

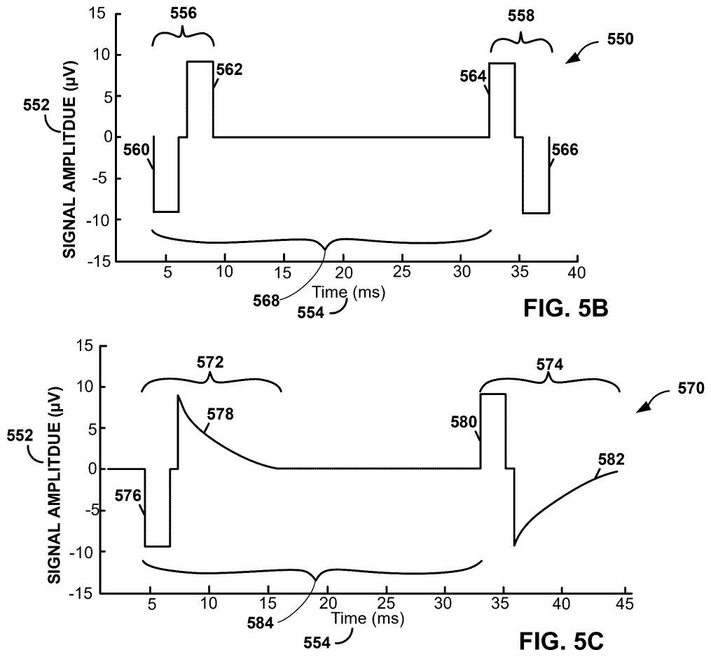

Each of the plurality of pulses may delivered as active recharge pulse such as active recharge pulse 556 or active recharge pulse 558 of active recharge stimulation 550 of FIG. 5B below, each of the plurality of pulses may delivered as passive recharge pulses such as passive recharge pulse 572 or passive recharge pulse 574 of passive recharge stimulation 570 of FIG. 5C below, or each of the plurality of pulses may delivered as some combination thereof. Each pulse may be a bi-phasic pulse that includes two phases of opposite polarity to remove the delivered charge from the tissue at the end of the pulse. For example, the plurality of pulses may be delivered in an active recharge setting, wherein stimulation generation circuitry 202 may control a first electrode and a second electrode of electrodes 116, 118, or a can of IMD 106, to deliver a first pulse, where the first pulse includes a cathodic-leading phase driven from the first electrode with an anodic phase driven from the second electrode with equal magnitude thereby forming an active recharge pulse. Thereafter stimulation generation circuitry 202 may deliver a second pulse, where the second pulse may be a second cathodic-leading pulse, which may be substantially similar to the first cathodic-leading pulse. Stimulation generation circuitry 202 may be configured to deliver active recharge biphasic stimulation. In some examples, the first pulse may be an anodic-leading pulse and the second pulse may be a cathodic-leading pulse. In some examples, the stimulation generation circuitry 202 may deliver the second pulse via the second electrode before delivering the first pulse via the first electrode. During therapy delivery, stimulation generation circuitry 202 may deliver only the first phase, omitting the second phase, and stimulation generation circuitry 202 may passively recharge after delivering the first phase, i.e., passive recharge stimulation 570. In some examples, the first electrode may be proximate to the second electrode. In some examples, the first electrode may be distal to the second electrode. The stimulation generation circuitry 202 may be configured to deliver passive recharge stimulation.

In some examples, stimulation generation circuitry 202 may be configured to deliver one or more of active recharge biphasic stimulation, tripolar stimulation, masker-probe stimulation, and/or alternating polarity stimulation. Stimulation is configured with a set of stimulation parameters of interest, including stimulating electrode configuration, pulse width, frequency, interphase interval and magnitude. Sensing is configured with a set of sensing parameters of interest, including sensing electrode configuration, blanking parameters, evoked response window (time after a stimulation pulse when the evoked response is expected to occur), and baseline window (time when an evoked response is not expected to occur to measure the baseline noise of the system).

The plurality of pulses may include 14 pulses, 20 pulses, 30 pulses, 40 pulses, 50 pulses, 60 pulses, 70 pulses, or any other number of pulses suitable to collect statistically sufficient data to average. In some examples, the selected magnitude may be constant for all pulses of the plurality of pulses. The selected magnitude may be a magnitude of the stimulation therapy which is delivered. The selected magnitude may be 0.5 mA, 0.3 mA, 0.4 mA, 0.6 mA, 0.7 mA, 1 mA, 2 mA, 5 mA, 10 mA or any other magnitude suitable to elicit one or more responses. In other examples, the selected magnitude may vary between one or more pulses of the plurality of pulses. In some examples, the selected magnitude may be a stepped ramp wherein a first set of the plurality of pulses has a first magnitude, a second set of the plurality of pulses has a second magnitude, and one or more other sets of the plurality of pulses has one or more other magnitudes. For example, the plurality of pulses may include five subsets of pulses where, a first sub-set of pulses may have a magnitude of 0.1 mA, a second sub-set of pulses may have a magnitude of 0.2 mA, a third sub-set of pulses may have a magnitude of 0.3 mA, a fourth sub-set of pulses may have a magnitude of 0.4 mA, and a fifth sub-set of pulses may have a magnitude of 0.5 mA. The pulses may be ordered from a lowest to a highest stimulation intensity. In some examples, the pulses may be divided into any number of subsets and the stimulation amplitude may vary between and within each of the subsets. In some examples, the selected magnitude may be a ramp where each pulse of the plurality of pulses has a higher magnitude than the pulse which directly preceded it in time.

Processing circuitry 210 may control sensing circuitry 204 to measure the electrical signals at pre-set times throughout the day and/or throughout the week. The pre-set times may be scheduled for the same time each day of the week to reduce variables in comparison. For example, the scheduled pre-set time may be during sleep of patient 112, e.g., 3 am each day, such that inter-day variations are reduced. The first cadence of sensing may be once a day, at a pre-set time each day. In some examples, the first cadence of sensing may be once a day, but sensing may occur at a random time throughout the day. In some examples, the first cadence may alternatively be four times a day, eight times a day, 12 times a day, or any other cadence between one and twelve times a day. In other examples, the first cadence may be 24 times a day, 96 times a day (i.e., once every 15 minutes), 288 times a day (i.e., once every 6 minutes), 1440 times a day (i.e., once a minute), or any other cadence as contemplated as suitable to balance the increased energy demand of more frequent measuring against the increased accuracy and representativeness of more frequent measurements.

User interface 302 of external device 104 may receive one or more inputs from a user, e.g., a patient, instructing processing circuitry 310 to control telemetry circuitry 308 to send a “therapy check” request to IMD 106. IMD 106 may receive the request via telemetry circuitry 208 which may instruct processing circuitry 210 to begin the process of measuring electrical signals at the first cadence (405) using any of the above methods.

After processing circuitry 210 has controlled stimulation generation circuitry 202 to deliver the plurality of pulses and processing circuitry 210 has controlled sensing circuitry 204 to sense the response to each, or some subset thereof, of the plurality of pulses, processing circuitry 210 may perform signal processing at a second cadence (415). In some examples, the signal processing may include the evaluation process of determining whether or not to adjust one or more parameter values based on the sensed signals from the first cadence. The signal processing may occur within an adjustment window or at an adjustment time that corresponds to the second cadence.

In some examples, the second cadence may be less frequent than the first cadence. In some examples, the second cadence may be once every five days. In some examples, the second cadence may be once every other day, once a day, twice a day, or four times a day. In some examples, the second cadence may be once every five days, once every seven days (i.e., once a week), once every 14 days, or once every 21 days. User interface 302 of external device 104 may receive one or more inputs from a user, e.g., a patient, instructing processing circuitry 310 to control telemetry circuitry 308 to send a “therapy check” request to IMD 106. IMD 106 may receive the request via telemetry circuitry 208 which may instruct processing circuitry 210 to begin the process of measuring electrical signals (405) using any of the above methods and directly thereafter perform signal processing (415).

In some examples, signal processing may include processing circuitry 210 time aligning all of the responses recorded by the sensing circuitry 204 to each of the plurality of pulses delivered by stimulation generation circuitry 202 based on a peak negative response. Processing circuitry 210 may thereafter sum or average all of the responses to each of the plurality of pulses. In some examples, processing circuitry 210 may average all of the responses recorded since the last signal processing step 415, which may be more than one sensing cycle as the first cadence may be equal to or shorter than the second cadence. Processing circuitry 210 may compare a peak negative value of the averaged response to a peak positive value of the response. The peak to trough value may thereafter be recorded as the signal magnitude for that therapy stimulation magnitude. In some examples, processing circuitry 210 may perform any other signal processing suitable to increase a signal to noise ratio of a signal.

The signal processing may include analog filtering. Analog filtering may include one or more analog filters which may be implemented in hardware. The one or more analog filters may be a high-pass filter (e.g., high frequency signals are selectively not attenuated and low frequency signals are selectively attenuated), a low-pass filter (e.g., low frequency signals are selectively not attenuated and high frequency signals are selectively attenuated), a bandstop filter (e.g., a frequency band is selectively attenuated and all other frequencies are selectively not attenuated) and a bandpass filter (e.g., a frequency band is selectively not attenuated whereas other frequencies are attenuated). The recorded signals may be digitized. The recorded signals may then be digitally filtered. Digital filtering may include one or more digital filters which may be implemented on processing circuitry 210. Digital filtering may include decimation (e.g., reducing the sampling rate of a signal by retaining only a subset of the samples, which may decrease data size and computational load while preserving essential information,) interpolation (e.g., increasing the sample rate of a signal by inserting additional sample points between existing ones, which may include upsampling followed by low-pass filtering to smooth out the resulting waveform), anti-aliasing (e.g., removing high-frequency components from a signal before sampling, as such the removed frequencies may not be misinterpreted as lower frequencies, which may thereby cause distortion in the digitized signal), and derivative (e.g., may include applying a convolution with derivative kernels, which may measure the rate of change of a signal and thereby enhance features such as rapid intensity change). A derivative filter may reduce artifacts and selectively enhance an evoked signal (e.g., an EMG and/or ECAP signal) while selectively attenuating lower frequency noise such as drifting. Additionally, the derivative filter may yield a higher amplitude, i.e., a larger slope, in a window which contains an evoked signal (e.g., an EMG and/or ECAP signal) than during a baseline signal. Additional Analog and Digital filtering may be performed as suitable to increase signal to noise ratios.

Filtering may additionally include techniques to mitigate the impact of stimulation artifact contamination. Processing circuitry 210 may perform template fitting and subtraction or summing of alternating polarity stimulation pulses. Summation of alternating polarity stimulation pulses may be substantially similar to active recharge stimulation 550 of FIG. 5B below and/or passive recharge stimulation 570 of FIG. 5C below. For example, summing of alternating stimulation pulses may include stimulation generation circuitry 202 controlling a first electrode of electrodes 116, 118 to deliver a first pulse, where the first pulse is a cathodic-leading pulse and includes an associated anode at a second electrode of electrodes 116, 118. Thereafter stimulation generation circuitry 202 may control the second electrode of electrodes 116, 118 to deliver a second pulse, where the second pulse may be a second anodic-leading pulse which has equal magnitude, where the first electrode is an associated anode. The sensed response to the first pulse may be summed with the sensed response to the second pulse. The first pulse may be delivered before the second pulse. The first electrode may be different than the second electrode. The first electrode or the second electrode may be a can of the medical device, and therefore the stimulation delivered may be monopolar stimulation. In some examples, three or more electrodes deliver the stimulation generated by the stimulation generation circuitry 202. The alternating stimulation pulses may be repeated for each of the electrodes of 116, 118.

Processing circuitry 210 may additionally mitigate other sources of noise, including electrical circuit noise, physiological noise (e.g., ECG, spontaneous myopotentials), and environmental noise (e.g., 60 Hz noise, EMI, movement artifact), through one or more analog and/or digital filters. Processing circuitry 210 may additionally mitigate outliers by determining one or more properties of a sensed window and comparing the determined one or more properties to a predetermined threshold. The one or more properties may include a power in a frequency band, a signal amplitude, and/or a peak/valley amplitude/latency. Processing circuitry 210 may determine a power in a frequency band by applying a fast Fourier transform (FFT) on a recorded signal to convert the signal from the time domain to the frequency domain. Processing circuitry 210 may then apply a bandpass filter to select a frequency band and thereafter may calculate the average of the frequency or alternatively processing circuitry 210 may calculate the power spectral density, which may include taking a squared magnitude of band passed frequency domain results. Processing circuitry 210 may compare the determined power to a threshold, wherein if the power does not meet the threshold requirements (i.e., the power of the frequency band is lower than the threshold and/or additionally or alternatively the power of a different frequency band is higher than the threshold), processing circuitry 210 may exclude the signal.

Processing circuitry 210 may calculate signal amplitude through an average of signal values to determine the signal amplitude, wherein processing circuitry 210 may exclude the signal based on the calculated signal amplitude. Processing circuitry 210 may calculate the peak to valley latency by determining a time at which the signal was at its lowest amplitude and comparing the time value at which the signal was at its highest amplitude. This comparison may yield a time between the peak and the valley of the recorded signal. Processing circuitry 210 may exclude the signal if the time between the peak and the valley is outside of an acceptable range. In some examples, the thresholds for the frequency power, the signal amplitude, and the peak/valley latency are pre-defined. In some examples, processing circuitry 210 may average one or more features over one or more windows and set the threshold for the frequency power, the signal amplitude, and the peak/valley latency based on the average over the multiple windows. Processing circuitry 210 may additionally or alternatively determine one or more population statistics of one or more features over one or more windows and set the threshold for the frequency power, the signal amplitude, and the peak/valley latency based on the population statistics. The population statistics may include a standard deviation from the mean. Processing circuitry 210 may determine if the sensed data of the window meets the criteria of an outlier, wherein processing circuitry 210 may exclude that data which meets the criteria of an outlier. Processing circuitry 210 may trigger stimulation generation circuitry 202 and sensing circuitry 204 to stimulate and thereafter record one or more additional recordings to replace the one or more data classified as an outlier.

Processing circuitry 210 may determine one or more features of interest from the processed signal data. Processing circuitry 210 may determine the one or more features on individual evoked response windows and associated baseline windows. In some examples, processing circuitry 210 determines the one or more features on an average of multiple evoked response or baseline windows. In some examples, processing circuitry 210 determines the one or more features on a sum of multiple evoked response or baseline windows. Processing circuitry 210 may calculate features such as an amplitude of the signal (e.g., peak-to-peak amplitude, amplitude of peaks and valleys, envelope, etc.), temporal properties (e.g., latency of peaks and valleys, width of peaks and valleys, phase, etc.), features from transformation of the signals (e.g., Fourier, wavelet, Hilbert, etc.), template comparison, features from dimensionality reduction techniques (e.g., principal component analysis, independent component analysis, etc.), or features from population statistics of from multiple sensed windows (e.g., standard deviation of peak-to-peak amplitude across a set of signals).

Processing circuitry 210 may determine a peak-to-peak amplitude of the signal by comparing a peak of a first signal, such as a first evoked signal response, to a peak of a signal, such as a second evoked signal response. Processing circuitry 210 may determine a peak to valley response by comparing a peak of a first signal to a valley of a first signal, wherein the first signal may be a first evoked response. Processing circuitry 210 may calculate the peak to valley by determining a time at which the signal was at its lowest amplitude and comparing the time value at which the signal was at its highest amplitude. This comparison may yield a time between the peak and the valley of the recorded signal. Processing circuitry 210 may calculate a peak (or valley) width by determining a first time at which the signal crosses a set threshold, determining a second when the signal cross the set threshold again, and comparing the first time and the second time. This comparison may yield a time width of a peak or a valley.

Processing circuitry 210 may additionally or alternatively calculate a Fourier transform of the signals (e.g., the evoked response, the baseline response, and/or the evoked response modulated by the baseline response). The Fourier transform may be the result of a fast Fourier transform. Processing circuitry 210 may determine one or more features based on a calculation of a wavelet transform of the signals. Processing circuitry 210 may determine one or more features based on a calculation of a Hilbert transform of the signals. Processing circuitry 210 may determine the one or more features using template comparison where processing circuitry may calculate the correlation of an evoked response to a one or more known signal templates. Processing circuitry 210 may subtract or otherwise modulate the evoked response based on the template to determine a response compared to the template which may be an expected response format.

Processing circuitry 210 may determine the one or more features through a machine learning or principal component analysis methods. A machine learning or artificial intelligence algorithm may operate by learning patterns from data to make predictions or decisions without being explicitly programmed. The process may begin with the collection of a training dataset, which is a large set of labeled data, wherein labelled data are points of data which were labelled by a human or another AI system such that each input data set is associated with an outcome. (e.g., data sets may be associated with desirable or undesirable implantation locations.) The training dataset may be used to train the machine learning model to learn the relationships between input features and the corresponding output labels. The model may adjust internal parameters to minimize predication errors. This phase involves techniques such as gradient descent and backpropagation in neural networks, where the model iteratively improves prediction accuracy by comparing model predictions against the actual labels and updating parameters accordingly. The neural network may include one or more input nodes, one or more hidden nodes, and one or more output nodes.

The model may be evaluated for performance after training using unseen data. This is where a test dataset may be used to test the performance of the model. The test dataset may be a separate set of data that the model has not encountered during training. By evaluating the model on the test dataset, the model's performance in real-world scenarios may be determined. Metrics such as accuracy, precision, recall, and F1-score are commonly used to measure the model's performance. If the model performs well on the test dataset, then the model has likely successfully learned the underlying patterns in the training data and may generalize to new data. In contrast, poor performance of model may indicate issues such as overfitting, where the model has learned the training data too well, including noise and outliers, and fails to generalize. In such cases, techniques like cross-validation, regularization, or gathering more diverse training data might be employed to improve the model's robustness and performance. It is well understood that machine learning and neural networks cannot be performed in the mind of a human being due to the computation complexity and timeliness requirements of such data association tasks.

Principal component analysis may include processing circuitry 210 determining one or more principal components such as eigenvectors. Principal Component Analysis (PCA) may be a technique to reduce the dimensionality of data while preserving variability of the data. PCA may include identifying the directions (principal components) along which the data varies the most. Independent component analysis (ICA) may be used in some examples. Principal components of the recorded data may be determined by eigenvectors of a covariance matrix of the data. The corresponding eigenvalues may indicate a magnitude of variance along each of the eigenvectors. PCA may transform the original data into a new set of uncorrelated variables by projecting the data onto the eigenvectors with the largest eigenvalues, thereby simplifying the dataset while retaining its essential patterns.

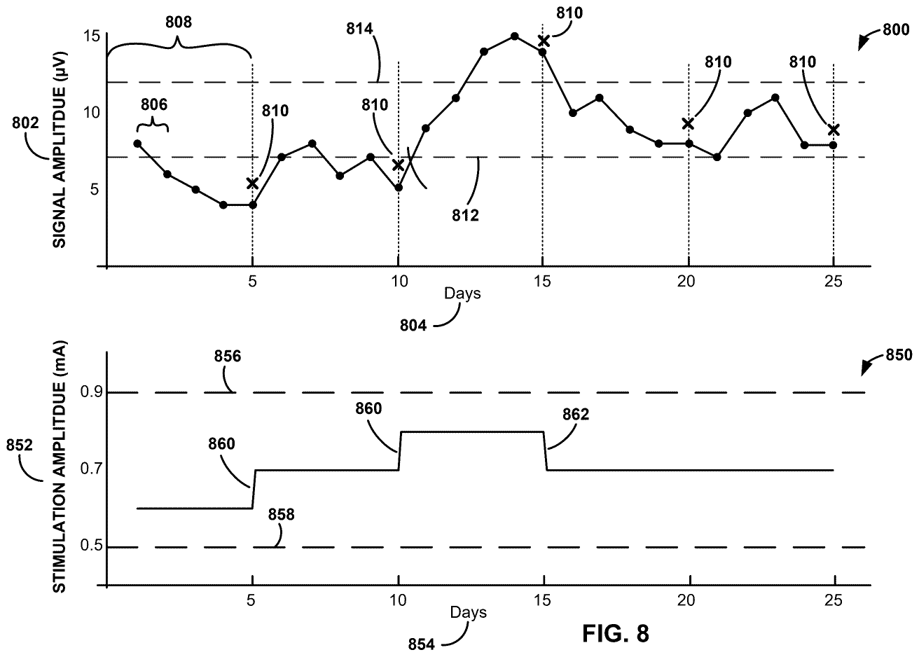

Processing circuitry 210 may determine whether the signals from the signal processing are outside of a signal parameter range (420). If the signal parameters are not outside of the signal parameter range, “NO” branch of block 420 then the processing circuitry 210 does not recommend any changes to the therapy parameters. If the signal parameters are outside of the signal parameter range, “YES” branch of block 420 then the processing circuitry 210 does recommend changes to the therapy parameters. An example of the signal parameter range and the recommendation to or not to change may be seen below in FIG. 8.

The signal parameter range may be a range of any selected parameters. The signal parameter range may be pre-set by a manufacturer based on typical, expected, ranges for the signals. In some examples, the signal parameter range may be set or modified by the clinician for the individual. In some examples, the signal parameter range may be a range of signal magnitudes. The signal magnitude range may be between 1 μV and 10 mV. The signal magnitude range may be between 1 μV and 100 μV, 10 μV and 50 μV, 100 μV and 250 μV, 1 mV and 10 mV, 100 μV and 1 mV, or any other range as determined by a clinician as an adequate response to stimulation. The signal parameter range may alternatively be based on a time duration of the response, a frequency profile (i.e., spectrogram) of the response, or any other characteristic of the response indicative of a suitability of the stimulation. Such parameters or characteristics of the signal may include a signal peak, a signal peak amplitude, a number of signal peaks, an area under signal peaks, a signal peak width, a time between signal peaks, a ratio of signal peak amplitudes, a ratio of signal peak widths, a ratio of areas under signal peaks, a latency of a signal peak, a signal valley, a signal valley amplitudes, a number of signal valleys, an area above a signal valley, a signal valley width, a time between signal valleys, a ratio of signal valley amplitudes, a ratio of signal valley widths, a ratio of areas above signal valleys, a valley latency, a root-mean-square signal value, a signal skew, a signal kurtosis, a signal frequency, a signal spectral content, a Hjorth feature, a signal amplitude growth curve threshold, a signal amplitude growth curve inflection point amplitude, a signal amplitude growth curve inflection point latency, a signal amplitude growth curve saturation point, a signal strength duration curve chronaxie, a signal strength duration curve rheobase, or another signal strength duration curve feature, a signal maximum rate of change feature (e.g., maximum of the derivative of the signal), or a signal minimum rate of change feature (e.g., the minimum of the derivative of the signal), or any other suitable signal feature.

Processing circuitry 210 may determine whether the signal is less than a signal parameter range (430). Processing circuitry 210 may compare the signals to the signal parameter range and if the signals are outside the signal parameter range and not less than the signal parameter range, “NO” branch of block 430, (i.e., greater than the signal parameter range) then processing circuitry 210 may decrease a stimulation intensity (435). Processing circuitry 210 may additionally compare the signals to the signal parameter range and if the signals are outside the signal parameter range and less than the range, “YES” branch of block 430, (i.e., less than the signal parameter range) then processing circuitry 210 may increase a stimulation intensity (440). An example of the signal parameter range and parameters that define when to adjust the parameter value may be seen below in FIG. 8.

If processing circuitry 210 determines that the signals are outside the signal parameter range and not less than (i.e., greater than) the signal parameter range, “NO” branch of block 430, then processing circuitry 210 may modify one or more parameters of the stimulation to decrease a stimulation intensity (435) applied via electrodes 116, 118 to patient 112. Processing circuitry 210 may compare the stimulation parameter to be decreased against a lower bound threshold for that parameter. If processing circuitry 210 determines that the stimulation parameter to be decreased is at a lower bound threshold for that parameter, processing circuitry 210 may notify a clinician instead of further decreasing the stimulation parameter. If processing circuitry 210 determines that the stimulation parameter to be decreased is not at a lower bound threshold for that parameter, processing circuitry 210 may decrease the stimulation parameter and resume therapy as normal.

If processing circuitry 210 determines that the signals are outside the signal parameter range and are less than the signal parameter range, “YES” branch of block 430, then processing circuitry 210 may modify one or more parameters of the stimulation to increase a stimulation intensity (440) applied via electrodes 116, 118 to patient 112. Processing circuitry 210 may compare the stimulation parameter to be increased against an upper bound threshold for that parameter. If processing circuitry 210 determines that the stimulation parameter to be increased is at an upper bound threshold for that parameter, processing circuitry 210 may notify a clinician instead of further increasing the stimulation parameter. If processing circuitry 210 determines that the stimulation parameter to be increased is not at an upper bound threshold for that parameter, processing circuitry 210 may increase the stimulation parameter and resume therapy as normal. Processing circuitry 210 may be configured to adjust any stimulation parameter including stimulating electrode configuration, sensing electrode configuration, pulse width, frequency, interphase interval and magnitude. Whether processing circuitry 210 increases, decreases, or does not change the stimulation parameters, processing circuitry 210 returns to delivering therapy at the pre-set cadence and measuring the electrical signals at the first cadence as described above with regard to steps 400-410.

FIG. 5A is a flowchart illustrating an example operation of a device configured to process the sensed signal based on peak-to-peak averages. The example process of FIG. 5A will be described with respect to processing circuitry 210 of IMD 106, but other processing circuitry, such as processing circuitry 310 of external device 104 may additionally, or alternatively, perform at least some of the elements of the process.

Processing circuitry 210 may control stimulation generation circuitry 202 to deliver a plurality of evoking pulses (500), and processing circuitry 210 may control sensing circuitry 204 to sense the evoked response to each of the plurality of evoking pulses (505). In some examples, sensing circuity 204 may be configured to sense each evoked response after an immediately preceding respective evoking pulse (e.g., a pulse of test stimulation). In this manner, the steps of 500 and 505 may actually be repeated in a cycle for as many evoking pulses and respective evoked responses occur. Processing circuitry 210 may determine whether there is sufficient data (510). If there is not sufficient data “NO” branch of block 510, processing circuitry 210 may repeat steps 500, 505, and 510 until there is sufficient data. If there is sufficient data “YES” branch of block 510, processing circuitry 210 may analyze the evoked responses using peak to peak average analysis (520) and thereafter processing circuitry 210 may determine whether the data is in range (525) and if the data is not in range, “NO” branch of block 525, then processing circuitry 210 may adjust stimulation therapy (530) based on the analyzed evoked responses whereas if the data is in range, “YES” branch of block 525, then the processing circuitry 210 may return to step 500 and repeat steps 500-525 as suitable.

Processing circuitry 210 may control stimulation generation circuitry 202 to deliver a plurality of evoking pulses (500). Processing circuitry 210 of IMD 106 may control stimulation generation circuitry 202 to deliver a stimulation signal via a plurality of electrodes 116, 118 to a target tissue. Processing circuitry 210 may control stimulation generation circuitry 202 to deliver a first pulse of a plurality of pulses at a selected magnitude.

Each of the plurality of pulses may delivered as active recharge pulse such as active recharge pulse 556 or active recharge pulse 558 of active recharge stimulation 550 of FIG. 5B below, each of the plurality of pulses may delivered as passive recharge pulses such as passive recharge pulse 572 or passive recharge pulse 574 of passive recharge stimulation 570 of FIG. 5C below, or each of the plurality of pulses may delivered as some combination thereof. For example, the plurality of pulses may be delivered in an active recharge setting, wherein stimulation generation circuitry 202 may control a first electrode of electrodes 116, 118, or a can of IMD 106, to deliver a first pulse, where the first pulse includes a cathodic-leading phase driven from the first electrode with an anodic phase driven from the second electrode with equal magnitude thereby forming an active recharge pulse. Thereafter stimulation generation circuitry 202 may deliver a second pulse, where the second pulse may be a second cathodic-leading pulse which may be substantially similar to the first cathodic-leading pulse. Stimulation generation circuitry 202 may be configured to deliver active recharge biphasic stimulation, for example active recharge stimulation 550. In some examples, the first pulse may be an anodic-leading pulse and the second pulse may be a cathodic-leading pulse. In some examples, the stimulation generation circuitry 202 may deliver the second pulse via the second electrode before delivering the first pulse via the first electrode. During therapy delivery, stimulation generation circuitry 202 may deliver only the first phase, omitting the second phase, and stimulation generation circuitry 202 may passively recharge after delivering the first phase, i.e., passive recharge stimulation 570. In some examples, the first electrode may be proximate to the second electrode. In some examples, the first electrode may be distal to the second electrode. The stimulation generation circuitry 202 may be configured to deliver passive recharge stimulation, for example passive recharge stimulation 570.

In some examples, the selected magnitude may be constant for all pulses of the plurality of pulses. The selected magnitude may be an magnitude of the stimulation therapy which is delivered. The selected magnitude may be 0.5 mA, 0.3 mA, 0.4 mA, 0.6 mA, 0.7 mA or any other magnitude suitable to elicit one or more responses. In other examples, the selected magnitude may vary between one or more pulses of the plurality of pulses. In some examples, the selected magnitude may be a stepped ramp wherein a first set of the plurality of pulses has a first magnitude, a second set of the plurality of pulses has a second magnitude, and one or more other sets of the plurality of pulses has one or more other magnitude. For example, the plurality of pulses may include five subsets of pulses where, a first sub-set of pulses may have a magnitude of 0.1 mA, a second sub-set of pulses may have a magnitude of 0.2 mA, a third sub-set of pulses may have a magnitude of 0.3 mA, a fourth sub-set of pulses may have a magnitude of 0.4 mA, and a fifth sub-set of pulses may have a magnitude of 0.5 mA. The pulses may be ordered from a lowest to a highest stimulation intensity. In some examples, the pulses may be divided into any number of subsets and the stimulation amplitude may vary between and within each of the subsets. In some examples, the selected magnitude may be a ramp where each pulse of the plurality of pulses has a higher magnitude than the pulse which directly preceded it in time.

Processing circuitry 210 may control sensing circuitry 204 to sense the evoked response to each of the plurality of evoking pulses (505). Processing circuitry 210 of IMD 106 may control sensing circuitry 204 to measure electrical signals from a plurality of electrodes 116, 118. Processing circuitry 210 may control sensing circuitry 204 to sense the stimulation-evoked signals in response to the first pulse of the plurality of pulses at the selected magnitude. Processing circuitry 210 may control sensing circuitry 204 to measure the electrical signals at pre-set times throughout the day and/or throughout the week. The pre-set times may be scheduled for the same time each day of the week to reduce variables in comparison. For example, the scheduled pre-set time may be during sleep of patient 112, e.g., 3 am each day, such that inter-day variations are reduced.

Processing circuitry 210 may determine whether there is sufficient data (510). Processing circuitry 210 may determine whether there is sufficient data based on whether there is sufficient data for the second cadence, i.e., whether there is sufficient data to process the data. Since processing circuitry 210 checks whether there is sufficient data prior to processing the data, processing circuitry 210 may wait until the second cadence has completed prior to running the analysis. When processing circuitry 210 waits until after the second cadence to run the processing steps, processing circuitry 210 is saving energy in the energy constrained environment because it is not computing analyses and later recomputing analyses. Furthermore, processing circuitry 210 may not write the analyses to memory and may not store them for long periods of time which further saves energy and storage space.

If there is not sufficient data (“NO” branch of block 510), processing circuitry 210 may repeat steps 500, 505, and 510 until there is sufficient data. To obtain sufficient data, processing circuitry 210 may repeat steps 500, 505, and 510 for X additional responses (515). In some examples, whether sufficient data has been collected may be based on a passage of time. For example, processing circuitry 210 may collect a first number of responses at each first cadence. Once the first number of responses is collected, then processing circuitry 210 waits at step 515 until the completion of the first cadence where processing circuitry 210 may then collect another first number of responses. Processing circuitry 210 may repeat the process of collecting the first number of responses for each first cadence until a second cadence has passed. After the second cadence has passed, processing circuitry 210 continues to step 520. In other examples, whether sufficient data has been collected may be based on a number of collected response. For example, processing circuitry 210 may collect a first number of responses at each first cadence and processing circuitry 210 may continue to step 520 when processing circuitry 210 has collected a threshold number of responses.

If there is sufficient data (“YES” branch of block 510), processing circuitry 210 may analyze the evoked responses using peak to peak average analysis (520) and thereafter processing circuitry 210 may determine whether the data is in range (525) and if the data is not in range, “NO” branch of block 525, then processing circuitry 210 may adjust stimulation therapy (530) based on the analyzed evoked responses whereas if the data is in range, “YES” branch of block 525, then the processing circuitry 210 may return to step 500 and repeat steps 500-525 as suitable.

Analyzing the evoked responses using peak to peak average analysis (520) may include processing circuitry 210 time aligning all of the responses sensed by the sensing circuitry 204 in (505) to each of the plurality of pulses delivered by stimulation generation circuitry 202 in step (500) based on a peak negative portion of their response. Processing circuitry 210 may thereafter average all of the responses to each of the plurality of pulses. Processing circuitry 210 may compare a peak negative value of the averaged response to a peak positive value of the averaged response. The peak to trough value may thereafter be recorded as the signal magnitude for that therapy stimulation magnitude. In some examples, processing circuitry 210 may perform any other signal processing suitable to increase a signal to noise ratio of a signal. Based on the analysis, processing circuitry 210 may determine whether the signals from the signal processing are outside of a signal parameter range (525). Processing circuitry 210 may compare each of the one or more evoked responses and their respective analyzes to one or more threshold which may be a threshold range. The threshold range may be pre-set or may be adjusted based on one or more factors. Adjusting stimulation therapy (530) may include adjusting any one or more stimulation parameters, including stimulation magnitude. For example, if the sensed response magnitude is too low, processing circuitry 210 may increase one or more stimulation parameters. If the sensed response magnitude is too high, processing circuitry 210 may decrease one or more stimulation parameters. In some examples, determining whether the signals from the signal processing are outside of a signal parameter range (525) may be analogous to steps 415-430 of FIG. 4 while adjusting stimulation therapy (530) may be analogous to steps 430-440 of FIG. 4.

FIG. 5B is graph of an example stimulation employing active recharge. FIG. 5C is graph of an example stimulation employing passive recharge. FIGS. 5B and 5C will be discussed together. The example stimulations of FIGS. 5B and 5C will be described with respect to processing circuitry 210 and related components of IMD 106, but other processing circuitry and related components, such as processing circuitry 310 of external device 104 may additionally, or alternatively, be employed. Active recharge stimulation 550 and passive recharge stimulation 570 illustrate examples of stimulation that processing circuitry 210 may control stimulation generation circuitry 202 to deliver through electrodes 116, 118 of IMD 106. Signal amplitude 552 is measured in microvolts (μV) and are for exemplary purposes as any suitable range of stimulation amplitude, sufficient to activate one or more muscles, may be used. Time axis 554 represents a time over which the signals are delivered. Active recharge alternating polarity stimulation 550 may include first pulse 556 and

second pulse 558. In some examples, active recharge stimulation 550 may include any number of pulses suitable to deliver a test stimulation to a patient. In some examples, active recharge stimulation 550 includes 14 pulses, 20 pulses, 30 pulses, 40 pulses, 50 pulses, 60 pulses, 70 pulses, or any other suitable number of pulses to achieve a sufficient signal to noise ratio to determine whether the data is in a suitable range. First pulse 556 may include first cathodic phase 560 and first anodic phase 562 separated by an interphase interval. Processing circuitry 210 may control stimulation generation circuitry 202 to deliver first cathodic phase 560 as pulse of negative amplitude for a first duration, pause for a period of time shorter than the first duration, then processing circuitry 210 may control stimulation generation circuitry 202 to deliver first anodic phase 562 of positive amplitude for the first duration or for a second duration suitable to reduce the built up charge from first cathodic phase 560. In some examples, the area under the curve, i.e., the amount of charge delivered, by first cathodic phase 560 and first anodic phase 562 may be equal. Processing circuitry 210 may control sensing circuitry 204 to record from electrode 116, 118 after controlling stimulation generation circuitry 202 to deliver first anodic phase 562. Processing circuitry 210 may wait for a period such as period 568. Period 568 may be the inverse of the frequency at which pulses are delivered. In some examples, the frequency may be 2 kHz or any other frequency suitable to deliver one or more test pulses.

After period 568, processing circuitry 210 may deliver second pulse 558. Second pulse 558 may include second anodic phase 564 and second cathodic phase 566 separated by an interphase interval. To deliver second pulse 558, processing circuitry 210 may control stimulation generation circuitry 202 to deliver second anodic phase 564 as pulse of positive amplitude for a first duration, pause for a period of time shorter than the first duration, then processing circuitry 210 may control stimulation generation circuitry 202 to deliver second cathodic phase 566 of negative amplitude for the first duration or for a second duration suitable to reduce the built up charge from first cathodic phase 560. In some examples, the area under the curve, i.e., the amount of charge delivered, by second cathodic phase 564 and second anodic phase 566 may be equal. In other examples, first pulse 556 and second pulse 558 may be flipped. In other examples, first pulse 556, or second pulse 558, may be repeated one or more additional times. In some examples, first pulse 556 and second pulse 558 may both be anodic-leading pulses (i.e., both 560 and 564 are anodic phases with 562 and 566 being cathodic phases) or may both be cathodic-leading pulses (i.e., both 560 and 564 are cathodic phases with 562 and 566 being anodic phases).

Passive recharge stimulation 570 may include first pulse 572 and second pulse 574. In some examples, passive recharge stimulation 570 may include any number of pulses suitable to deliver a test stimulation to a patient. In some examples, active recharge alternating polarity stimulation 570 includes 14 pulses, 20 pulses, 30 pulses, 40 pulses, 50 pulses, 60 pulses, 70 pulses, or any other suitable number of pulses to achieve a sufficient signal to noise ratio to determine whether the data is in a suitable range. First pulse 572 may include first cathodic phase 576 and first passive recharge phase 578 separated by an interphase interval. Processing circuitry 210 may control stimulation generation circuitry 202 to deliver first cathodic phase 576 as pulse of negative amplitude for a first duration, pause for a period of time shorter than the first duration, then processing circuitry 210 may control stimulation generation circuitry 202 to accept charge from the electrodes at first passive recharge phase 578 for a second duration suitable to reduce the built-up charge from first cathodic phase 576. In some examples, the area under the curve, i.e., the amount of charge delivered or received, by first cathodic phase 576 and first passive recharge phase 578 may be equal. Processing circuitry 210 may control sensing circuitry 204 to record from electrode 116, 118 after controlling stimulation generation circuitry 202 to passively accept charge during first passive recharge phase 578. Processing circuitry 210 may wait for a period such as period 584. Period 584 may be the inverse of the frequency at which pulses are delivered. In some examples, the frequency may be 2 kHz or any other frequency suitable to deliver one or more test pulses.

After period 584, processing circuitry 210 may deliver second pulse 574. Second pulse 574 may include anodic phase 580 and second passive recharge phase 582 separated by an interphase interval. To deliver second pulse 574, processing circuitry 210 may control stimulation generation circuitry 202 to deliver anodic phase 580 as pulse of positive amplitude for a first duration, pause for a period of time shorter than the first duration, then processing circuitry 210 may control stimulation generation circuitry 202 to passively accept any built up charge for a second duration at second passive recharge phase 582 where the second duration is longer than the first duration and suitable to reduce the built up charge from anodic phase 580. In some examples, the area under the curve, i.e., the amount of charge delivered or received, by anodic phase 580 and second passive recharge phase 582 may be equal. In other examples, first pulse 572 and second pulse 574 may be flipped. In other examples, first pulse 572, or second pulse 574, may be repeated one or more additional times. In some examples, first pulse 572 and second pulse 574 may both be anodic-leading pulses (i.e., both 576 and 580 are anodic phases) or may both be cathodic-leading pulses (i.e., both 576 and 580 are cathodic phases).

FIG. 6 is a flowchart illustrating an example operation of a device configured to sense one or more signals after exiting a standby mode and/or before entering the standby mode. The example process of FIG. 6 will be described with respect to processing circuitry 210 of IMD 106, but other processing circuitry, such as processing circuitry 310 of external device 104 may additionally, or alternatively, perform at least some of the elements of the process.

A clinician may activate the implantable medical device (600), the implantable medical device may wait in a standby mode for a set period (605), optionally the implantable medical device may deliver one or more test stimulations and sense (610), the implantable medical device may deliver therapy stimulation (615), thereafter optionally the implantable medical device may deliver one or more test stimulations and sense (620), and the implantable medical device may enter a standby mode (625).

A clinician may activate the implantable medical device (600). The implantable medical device may be activated with pre-set values provided by the manufacturer wherein the physician accepts the pre-set values or adjusts one or more of the parameters. In some examples, the pre-set values may include magnitude limits for stimulation, sensed signal magnitude range, one or more stimulation parameters and may be adjusted based by any of the adjustment steps described herein. In some examples, one or more stimulation parameters may be individualized to the patient wherein a default value may have a range and the acceptable range may be absolute or percentage based. For example, if a signal threshold was 0.5 mA, the range may be +/−0.2 mA (i.e., 0.3-0.7 mA) or may additionally or alternatively be percentage based of +−/ 20% (i.e., 0.3-0.7 mA). The default values, acceptable ranges, absolute or percentage limits may be set by a manufacturer, a clinician, a program described herein, the patient, or any other suitable setting. Alternatively this activation step may be performed automatically or otherwise cause IMD 106 to enter an operational mode for a patient.

Processing circuitry 210 of IMD 106 may initially wait in a standby mode for a set period (605). In some examples, the set period may be the duration between scheduled delivery times for electrical stimulation therapy. The set period may be based on a first cadence wherein IMD 106 may wait in the standby period for a duration associated with the first cadence. After the duration of the first cadence passes, processing circuitry 210 may control IMD 106 to exit the standby mode and enter an operational mode. The operational mode may be a higher power state than the standby mode (which may have a low power state to conserve power). In the operational mode, IMD 106 may be configured to perform various tasks, such as deliver electrical stimulation therapy, deliver test stimulation and sense for evoked signals elicited by the test stimulation, and/or adjust one or more stimulation parameters based on the sensed evoked signals. Upon exiting the standby mode, processing circuitry 210 may control stimulation generation circuitry 202 to deliver one or more test stimulations and processing circuitry 210 may control sensing circuitry 204 to sense an evoked response to the test stimulations (610). In this manner, step 610 may occur within a sensing window. Step 610 may be optional in that it may be scheduled to be performed in every instance before step 615 or less frequently than step 615. Steps 610 and 620 may both be performed, or only one of steps 610 or 620 may be performed in any given loop of FIG. 6. In some examples, step 610 may be substantially similar to steps 500-515 of FIG. 5A.

After completing step 610, processing circuitry 210 may control stimulation generation circuitry 202 of IMD 106 to deliver therapy stimulation (615). In some examples, processing circuitry 210 may control stimulation generation circuitry 202 to deliver therapy stimulation at a therapy stimulation level for a therapy stimulation period. The therapy stimulation level may be controlled by processing circuitry 210 and may be adjusted based on the test stimulations and response as seen in steps 400-440 of FIG. 4 and/or steps 500-525 of FIG. 5A. The therapy stimulation period may be pre-set by a clinician and/or may be controlled by processing circuitry 210 and may be adjusted based on the test stimulations and response as seen in steps 400-440 of FIG. 4 and/or steps 500-525 of FIG. 5A. In some examples, the therapy stimulation period may be continuous (e.g., a continuous pulse train or repeating pattern of pulses). In some examples, the therapy stimulation period may be 30 minutes once a day. In some examples, the therapy stimulation period may be one hour once a day, two hours once a day, all day, or any other length or proportion of time suitable to delivering therapy to patient 112.

Optionally, and according to predetermine instructions, prior to entering the standby mode, processing circuitry 210 may control stimulation generation circuitry 202 to deliver one or more test stimulations and processing circuitry 210 may control sensing circuitry 204 to sense a response to the test stimulations (620). In some examples, (620) may be substantially similar to steps 500-515 of FIG. 5A. As discussed above, step 620 may be performed in addition to step 610 or as an alternative to step 610. Benefits to performing the sensing window during the operational mode together with stimulation delivery, as opposed to a separate standalone sensing window, may include battery efficiency of entering the operational mode fewer times.

The implantable medical device may enter a standby mode (625). Standby mode may occur for an inverse proportion of time to the therapy stimulation period. For example, if the therapy stimulation period is 30 minutes a day, the standby period may be 23 hours and 30 minutes a day. Processing circuitry 210 may control IMD 106 to exit the standby mode based on a temporal period passing, such as the first cadence passing. Telemetry circuitry 208 may instruct processing circuitry 210 to control IMD 106 to exit the standby mode based on a request from user interface 302 of external device 104. For example, a user of external device 104 may interact with user interface 302 of external device 104 to request a “therapy check” which may thereby request processing circuitry 210 to control IMD 106 to exit the standby mode and perform step 610 and/or step 620 with processing and adjustment, i.e., steps 415-440 of FIG. 4 and steps 520-525 of FIG. 5A. An advantage of standby mode is that power consumption is significantly reduced and therefore for the standby period, power consumption from power source 220 is significantly reduced. Reducing power consumption is beneficial because it may increase a length of time which IMD 106 may be implanted in patient 112 prior to requiring replacement (i.e., explanation and re-implantation). After entering the standby mode, processing circuitry 210 may control IMD 106 to return to step 605 and wait in the standby mode for the set period of time.

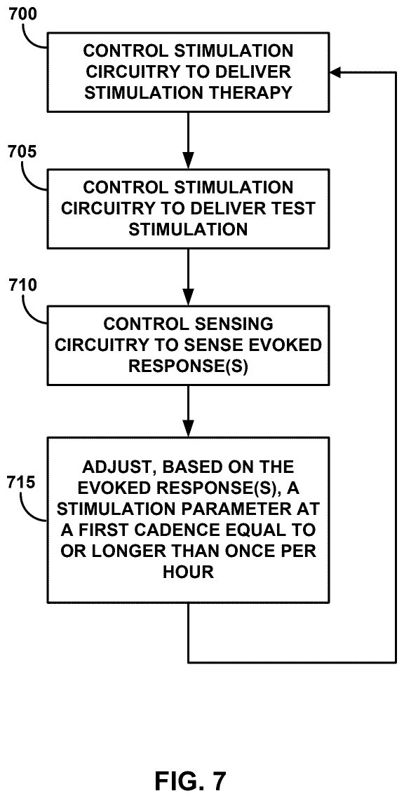

FIG. 7 is a flowchart illustrating an example operation of a device configured to adjust therapy less than once an hour after comparing evoked responses to baseline responses. The process of FIG. 7 will be described with respect to the components of IMD 106, such as processing circuitry 210, sensing circuitry 204, and stimulation circuitry 202. However, other device, or combinations of devices, may perform the techniques of FIG. 7.

As shown in the example of FIG. 7, processing circuitry 210 may control stimulation circuitry 202 to deliver therapy stimulation according to one or more stimulation parameters that define the therapy stimulation (700). Processing circuitry 210 may also control stimulation circuitry 202 to deliver test stimulation via a plurality of electrodes (705). The therapy stimulation may be different than the test stimulation. Processing circuitry 210 may then control sensing circuitry 204 to sense an evoked response elicited by the test stimulation during a sensing window following the test stimulation (710). In some examples, processing circuitry 210 may perform the steps 705 and 710 in multiple repeating iterations in order to generate multiple evoked signals that may be averaged or otherwise processed to obtain a more robust signal. In some examples, the test stimulation of step 705 and sensing of step 710 may occur prior to stimulation therapy delivery of step 700 in this loop.

Processing circuity 210 may then adjust, based on the evoked response(s), the therapy stimulation at a first cadence (715). The first cadence may correspond to the frequency with which processing circuitry 210 is configured to adjust one or more stimulation parameter values defining the stimulation therapy. In general, the first cadence of step 715 is equal to or longer than once per hour, but may be equal to or longer than once per day, once per two days, once per five days, etc. In this manner, processing circuitry 210 may perform the steps of 700-710 several times before processing circuitry 210 is configured to adjust the stimulation parameter value(s), if needed according to the evoked signals that were collected. The first cadence of step 715 may be equal to, or slower than, a second cadence at which processing circuitry 210 performs the steps of 705 and 710 during a sensing window. This relatively slower closed-loop therapy may be suitable for various stimulation schedules in which stimulation is delivered less frequently, such as less than an hour per day. The first cadence of 715 may be analogous to the second cadence of step 415 of FIG. 4 and the second cadence of 700-710 may be analogous to the first cadence of step 400 of FIG. 4. For example, the first cadence of 715 may be once every five days. In some examples, the first cadence of 715 may be once every other day, once a day, twice a day, or four times a day. In some examples, the first cadence of 715 may be once every five days, once every seven days (i.e., once a week), once every 14 days, or once every 21 days. User interface 302 of external device 104 may receive one or more inputs from a user, e.g., a patient, instructing processing circuitry 310 to control telemetry circuitry 308 to send a “therapy check” request to IMD 106. IMD 106 may receive the request via telemetry circuitry 208 which may instruct processing circuitry 210 to begin the process of measuring electrical signals steps 700-710 using any of the above methods and directly thereafter adjust a stimulation parameter according to step 715 using any of the methods described herein.

The second cadence of 700-710 may be once a day, at a pre-set time each day. In some examples, the second cadence of 700-710 of sensing may be once a day, but sensing may occur at a random time throughout the day. In some examples, the second cadence of 700-710 may alternatively be four times a day, eight times a day, 12 times a day, or any other cadence between one and twelve times a day. In other examples, the second cadence of 700-710 may be 24 times a day, 96 times a day (i.e., once every 15 minutes), 288 times a day (i.e., once every 6 minutes), 1440 times a day (i.e., once a minute), or any other cadence as contemplated as suitable to balance the increased energy demand of more frequent measuring against the increased accuracy and representativeness of more frequent measurements.

Processing circuity 210 may adjust therapy in response to one or more signals being out of a range and may instead not adjust therapy at all in response to one or more signals being in a range. As such, adjusting, based on the evoked response(s), the therapy stimulation at a first cadence (715) may be an optional step based on whether one or more signals are outside of a suitable range.

FIG. 8 is a graph of an example of sensed signals and adjusted stimulation magnitude over time. Sensed data 800 illustrates measurements (e.g., sensed evoked signals during a sensing window) obtained once a day. Analysis of the measurements occurs at a rate of once per five days, to include the last five days of data in that analysis. Stimulation magnitude 850 illustrates the signal magnitude over the course of the same period as sensed data 800.

Sensed data 800 illustrates signal output measurements being taken once a day from one or more electrodes. Signal magnitude 802 is measured in microvolts (μV). Signal magnitude 802 may be an amalgamation of one or more measurements as described in 405 of FIG. 4 and steps 500-515 of FIG. 5A. Days 804 may represent a time axis over which signals are recorded. First cadence 806 is a frequency at which processing circuitry 210 of IMD 106 controls sensing circuitry 204 to measure a signal magnitude at electrodes 116, 118. Second cadence 808 is a frequency at which processing circuitry 210 performs signal processing. Signal processing 810 are the times at which processing circuitry 210 performs the signal processing to determine whether the signal magnitude of an analysis period is greater than or less than a threshold. The analysis period may be equal to second cadence 808. In some examples, the analysis period is less than second cadence 808. In other examples, the analysis period is greater than second cadence 808. The threshold may be bounded by lower threshold 812 which sets a value, where if the recorded signal is below lower threshold 812, then processing circuitry 210 may increase the

stimulation magnitude. The threshold may be bounded by upper threshold 814 which sets a value, where if the recorded signal is above upper threshold 814, then processing circuitry 210 may decrease the stimulation magnitude.

Stimulation magnitude 850 illustrates therapy stimulation output from IMD 106 configured to deliver electrical stimulation to patient 112. Stimulation magnitude 852 represents a magnitude of the stimulation delivered by IMD 106 via electrodes 116, 118 to patient 112 via stimulation generation circuitry 202. Stimulation magnitude 852 may vary over the course of one or more days 854. Stimulation magnitude 852 may be bounded by stimulation upper threshold 856 above which magnitude IMD 106 may not automatically adjust above. Stimulation upper threshold 856 may be overridden by a clinician, Specifically, processing circuitry 210 may control telemetry circuitry 208 to request clinician to increase stimulation magnitude 852 beyond stimulation upper threshold 856. Stimulation magnitude 852 may be bounded by stimulation lower threshold 858 above which magnitude IMD 106 may not automatically adjust below. Stimulation lower threshold 858 may be overridden by a clinician, specifically, processing circuitry 210 may control telemetry circuitry 208 to request clinician to decrease stimulation magnitude 852 below stimulation lower threshold 858. Stimulation magnitude 852 may be increased 860 or decreased 862 at any of the one or more signal processing 810 points which occur at most every second cadence 808. In some examples, increase 860 and decrease 862 may be 0.1 mA increments. In other examples, increase 860 and decrease 862 may be any increment suitable to adjust the stimulation magnitude delivered by stimulation generation circuitry 201, including 0.05 mA increments, 0.075 mA increments, 0.125 mA increments, 0.2 mA increments, 0.3 mA increments, 0.4 mA increments, 0.5 mA increments, or 0.15 mA increments. Increase 860 and decrease 862 may be same magnitude. In some examples, increase 860 and decrease 862 may be different magnitudes.

The following examples are a non-limiting list of examples in accordance with one or more techniques of this disclosure.

Example 1: A system includes processing circuitry configured to: control stimulation circuitry to deliver a therapy stimulation and a test stimulation via a plurality of electrodes, wherein the therapy stimulation is different than the test stimulation; control sensing circuitry to sense an evoked response elicited by the test stimulation during a sensing window following the test stimulation; and adjust, based on the evoked response, the therapy stimulation at a first cadence, wherein the first cadence is equal to or longer than once per hour.

Example 2: The system of example 1, wherein a target location of the stimulation therapy comprises a sacral nerve of a patient, and wherein the plurality of electrodes are configured to be implanted in the patient.

Example 3: The system of any of examples 1 or 2, wherein the processing circuitry is configured to control the stimulation circuitry to deliver the test stimulation and sense the evoked response at a second cadence.

Example 4: The system of example 3, wherein the second cadence is equal to or longer than once per hour.

Example 5: The system of any of examples 3 or 4, wherein the first cadence is slower than the second cadence.

Example 6: The system of example 5, wherein the first cadence is equal to or longer than once every 5 days, and wherein the second cadence is equal to or longer than once per day.

Example 7: The system of any of examples 1 through 6, wherein the processing circuitry is configured to: control the stimulation circuitry to deliver the therapy stimulation for 30 minutes once a day; and control the sensing circuitry to sense the evoked response elicited by the test stimulation during a sensing window that occurs at least one of immediately before or immediately after the therapy stimulation is delivered.

Example 8: The system of any of examples 1 through 7, wherein the processing circuitry is configured to: control sensing circuitry to sense the evoked response elicited by the test stimulation by at least: controlling the stimulation circuitry to deliver at least 10 test stimulation pulses during the sensing window; and controlling the sensing circuitry to sense evoked responses from respective test stimulation pulses during the sensing window; determine an average evoked response based on the evoked responses; and adjust the therapy stimulation based on the average evoked response.

Example 9: The system of any of examples 1 through 8, wherein the therapy stimulation comprises passive recharge and the test stimulation comprises active recharge.

Example 10: The system of any of examples 1 through 8, wherein the therapy stimulation and the test stimulation comprise passive recharge.

Example 11: The system of any of examples 1 through 10, wherein the processing circuitry is configured to adjust the therapy stimulation by at least: comparing a characteristic of the evoked response to a threshold window, the threshold window comprising a first threshold and a second threshold, wherein the first threshold is lower than the second threshold; responsive to the characteristic being less than the first threshold, increasing an amplitude of the therapy stimulation; and responsive to the characteristic being greater than the second threshold, decreasing the amplitude of the therapy stimulation.

Example 12: The system of any of examples 1 through 11, further comprising an implantable medical device comprising the stimulation circuitry, the sensing circuitry, and the processing circuitry.

Example 13: A method includes controlling, by processing circuitry, stimulation circuitry to deliver a therapy stimulation and a test stimulation via a plurality of electrodes, wherein the therapy stimulation is different than the test stimulation; controlling, by the processing circuitry, sensing circuitry to sense an evoked response elicited by the test stimulation during a sensing window following the test stimulation; and adjusting, by the processing circuitry and based on the evoked response, the therapy stimulation at a first cadence, wherein the first cadence is equal to or longer than once per hour.

Example 14: The method of example 13, wherein a target location of the stimulation therapy comprises a sacral nerve of a patient, and wherein the plurality of electrodes are configured to be implanted in the patient.

Example 15: The method of any of examples 13 or 14, wherein the processing circuitry is configured to control the stimulation circuitry to deliver the test stimulation and sense the evoked response at a second cadence.

Example 16: The method of any of example 15, wherein the second cadence is equal to or longer than once per hour.

Example 17: The method of any of examples 15 or 16, wherein the first cadence is slower than the second cadence.

Example 18: The method of examples 17, wherein the first cadence is equal to or longer than once every 5 days, and wherein the second cadence is equal to or longer than once per day.

Example 19: The method of any of examples 13 through 18, further includes controlling the stimulation circuitry to deliver the therapy stimulation for 30 minutes once a day; and controlling the sensing circuitry to sense the evoked response elicited by the test stimulation during a sensing window that occurs at least one of immediately before or immediately after the therapy stimulation is delivered.

Example 20: The method of any of examples 13 through 19, wherein: controlling the sensing circuitry to sense the evoked response elicited by the test stimulation comprises: controlling the stimulation circuitry to deliver at least 10 test stimulation pulses during the sensing window; and controlling the sensing circuitry to sense evoked responses from respective test stimulation pulses during the sensing window; and wherein the method further comprises: determining an average evoked response based on the evoked responses; and adjusting the therapy stimulation based on the average evoked response.

Example 21: The method of any of examples 13 through 20, wherein the therapy stimulation comprises passive recharge and the test stimulation comprises active recharge.

Example 22: The system of any of examples 13 through 20, wherein the therapy stimulation and the test stimulation comprise passive recharge.

Example 23: The method of any of examples 13 through 22, wherein adjusting the therapy stimulation comprises: comparing a characteristic of the evoked response to a threshold window, the threshold window comprising a first threshold and a second threshold, wherein the first threshold is lower than the second threshold; responsive to the characteristic being less than the first threshold, increasing an amplitude of the therapy stimulation; and responsive to the characteristic being greater than the second threshold, decreasing the amplitude of the therapy stimulation.

Example 24: A non-transitory computer-readable storage medium includes control stimulation circuitry to deliver a therapy stimulation and a test stimulation via a plurality of electrodes, wherein the therapy stimulation is different than the test stimulation; control sensing circuitry to sense an evoked response elicited by the test stimulation during a sensing window following the test stimulation; and adjust, based on the evoked response, the therapy stimulation at a first cadence, wherein the first cadence is equal to or longer than once per hour.

Example 25. The system of any of Examples 1-24, wherein the processing circuitry is configured to control the stimulation circuitry to deliver the therapy stimulation to a target location comprising at least one of a pudendal nerve or a tibial nerve of the patient, and wherein the plurality of electrodes are configured to be implanted in a patient to deliver the therapy stimulation to the at least one of the pudendal nerve or the tibial nerve.

Example 26. A system comprising at least one memory configured to store instructions, and processing circuitry in communication with the at least one memory, the processing circuitry configured to control stimulation circuitry to deliver a therapy stimulation via a plurality of electrodes to a target location comprising a sacral nerve of a patient, wherein the plurality of electrodes are configured to be implanted in the patient, and wherein the therapy stimulation comprises passive recharge, control the stimulation circuitry to deliver a test stimulation at a second cadence, wherein the test stimulation comprises active recharge, control sensing circuitry to sense an evoked response elicited by the test stimulation during a sensing window following the test stimulation, and adjust, based on the evoked response, the therapy stimulation at a first cadence.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various examples have been described. These and other examples are within the scope of the following claims.

What is claimed is:

1. A system comprising:
processing circuitry configured to:
control stimulation circuitry to deliver a therapy stimulation and a test stimulation via a plurality of electrodes, wherein the therapy stimulation is different than the test stimulation;
control sensing circuitry to sense an evoked response elicited by the test stimulation during a sensing window following the test stimulation; and
adjust, based on the evoked response, the therapy stimulation at a first cadence, wherein the first cadence is equal to or longer than once per hour.

2. The system of claim 1, wherein a target location of the therapy stimulation comprises a sacral nerve of a patient, and wherein the plurality of electrodes are configured to be implanted in the patient.

3. The system of claim 1, wherein the processing circuitry is configured to control the stimulation circuitry to deliver the therapy stimulation to a target location comprising at least one of a pudendal nerve or a tibial nerve of the patient, and wherein the plurality of electrodes are configured to be implanted in a patient to deliver the therapy stimulation to the at least one of the pudendal nerve or the tibial nerve.

4. The system of claim 1, wherein the processing circuitry is configured to control the stimulation circuitry to deliver the test stimulation and sense the evoked response at a second cadence.

5. The system of claim 4, wherein the second cadence is equal to or longer than once per hour.

6. The system of claim 5, wherein the first cadence is slower than the second cadence.

7. The system of claim 6, wherein the first cadence is equal to or longer than once every 5 days, and wherein the second cadence is equal to or longer than once per day.

8. The system of claim 1, wherein the processing circuitry is configured to:
control the stimulation circuitry to deliver the therapy stimulation for 30 minutes once a day; and
control the sensing circuitry to sense the evoked response elicited by the test stimulation during a sensing window that occurs at least one of immediately before or immediately after the therapy stimulation is delivered.

9. The system of claim 1, wherein the processing circuitry is configured to:
control sensing circuitry to sense the evoked response elicited by the test stimulation by at least:
controlling the stimulation circuitry to deliver at least 10 test stimulation pulses during the sensing window; and
controlling the sensing circuitry to sense evoked responses from respective test stimulation pulses during the sensing window;
determine an average evoked response based on the evoked responses; and
adjust the therapy stimulation based on the average evoked response.

10. The system of claim 1, wherein the therapy stimulation comprises passive recharge and the test stimulation comprises active recharge.

11. The system of claim 1, wherein the processing circuitry is configured to adjust the therapy stimulation by at least:
comparing a characteristic of the evoked response to a threshold window, the threshold window comprising a first threshold and a second threshold, wherein the first threshold is lower than the second threshold;
responsive to the characteristic being less than the first threshold, increasing an amplitude of the therapy stimulation; and
responsive to the characteristic being greater than the second threshold, decreasing the amplitude of the therapy stimulation.

12. The system of claim 1, further comprising an implantable medical device comprising the stimulation circuitry, the sensing circuitry, and the processing circuitry.

13. A method comprising:
controlling, by processing circuitry, stimulation circuitry to deliver a therapy stimulation and a test stimulation via a plurality of electrodes, wherein the therapy stimulation is different than the test stimulation;
controlling, by the processing circuitry, sensing circuitry to sense an evoked response elicited by the test stimulation during a sensing window following the test stimulation; and
adjusting, by the processing circuitry and based on the evoked response, the therapy stimulation at a first cadence, wherein the first cadence is equal to or longer than once per hour.

14. The method of claim 13, wherein a target location of the therapy stimulation comprises a sacral nerve of a patient, and wherein the plurality of electrodes are configured to be implanted in the patient.

15. The method of claim 13, wherein the processing circuitry is configured to control the stimulation circuitry to deliver the test stimulation and sense the evoked response at a second cadence wherein the second cadence is equal to or longer than once per hour, and wherein the first cadence is slower than the second cadence, and wherein the first cadence is equal to or longer than once every 5 days, and wherein the second cadence is equal to or longer than once per day.

16. The method of claim 13, further comprising:
controlling the stimulation circuitry to deliver the therapy stimulation for 30 minutes once a day; and
controlling the sensing circuitry to sense the evoked response elicited by the test stimulation during a sensing window that occurs at least one of immediately before or immediately after the therapy stimulation is delivered.

17. The method of claim 13, wherein:
controlling the sensing circuitry to sense the evoked response elicited by the test stimulation comprises:
controlling the stimulation circuitry to deliver at least 10 test stimulation pulses during the sensing window; and
controlling the sensing circuitry to sense evoked responses from respective test stimulation pulses during the sensing window; and
wherein the method further comprises:
determining an average evoked response based on the evoked responses; and
adjusting the therapy stimulation based on the average evoked response.

18. The method of claim 13, wherein the therapy stimulation comprises passive recharge and the test stimulation comprises active recharge.

19. The method of claim 14, wherein adjusting the therapy stimulation comprises:
comparing a characteristic of the evoked response to a threshold window, the threshold window comprising a first threshold and a second threshold, wherein the first threshold is lower than the second threshold;
responsive to the characteristic being less than the first threshold, increasing an amplitude of the therapy stimulation; and
responsive to the characteristic being greater than the second threshold, decreasing the amplitude of the therapy stimulation.

20. A system comprising:
at least one memory configured to store instructions; and
processing circuitry in communication with the at least one memory, the processing circuitry configured to:
control stimulation circuitry to deliver a therapy stimulation via a plurality of electrodes to a target location comprising a sacral nerve of a patient, wherein the plurality of electrodes are configured to be implanted in the patient, and wherein the therapy stimulation comprises passive recharge;
control the stimulation circuitry to deliver a test stimulation at a second cadence, wherein the test stimulation comprises active recharge;
control sensing circuitry to sense an evoked response elicited by the test stimulation during a sensing window following the test stimulation; and
adjust, based on the evoked response, the therapy stimulation at a first cadence.
